(19) United States
(12) Patent Application Publication
(10) Pub. No.: US 2016/0280789 A1
(43) Pub. Date: Sep. 29, 2016

(54) METHODS FOR TREATING HEMATOLOGICAL MALIGNANCIES

(71) Applicants: CHILDREN'S HOSPITAL LOS ANGELES, Los Angeles, CA (US); FRED HUTCHINSON CANCER RESEARCH CENTER, Seattle, WA (US)

(72) Inventors: Yong-mi Kim, La Crescenta, CA (US); Elizabeth Wayner, Woodway, WA (US)

(73) Assignees: Children's Hospital Los Angeles, Los Angeles, CA (US); Fred Hutchinson Cancer Research Center, Seattle, WA (US)

(21) Appl. No.: 15/033,879

(22) PCT Filed: Nov. 10, 2014
(86) PCT No.: PCT/US14/64881

§ 371 Date: May 2, 2016

Related U.S. Application Data
(60) Provisional application No. 61/902,064, filed on Nov. 8, 2013.

(51) Int. Cl. C07K 16/28; A61K 47/48; A61K 39/395; A61K 45/06

(52) U.S. Cl. CPC C07K 16/2839; A61K 45/06; A61K 47/48561; A61K 39/39575; C07K 2317/76; C07K 2317/24; C07K 2317/622; A61K 2039/505

(57) ABSTRACT

Described herein are methods for treating hematological malignancies and/or solid tumors in a subject using inhibitors of integrin alpha 6. In some embodiments, the inhibitors are monoclonal antibodies. The antibodies may be conjugated to additional therapeutic agents. The antibodies may be co-administered sequentially or simultaneously with additional therapeutic agents.

### GOVERNMENT RIGHTS

The invention was made with government support under Grant No. CA172896 awarded by the National Institutes of Health. The government has certain rights to the invention.

### FIELD OF INVENTION

Provided are methods, compositions and kits for treating hematological malignancies in a subject in need thereof using an effective amount of an inhibitor of integrin alpha 6. The inhibitor may be a monoclonal antibody, for example, P5G10 or P1H8.

### BACKGROUND OF THE INVENTION

All publications cited herein are incorporated by reference in their entirety to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Minimal residual disease is a critical barrier in the treatment of acute lymphoblastic leukemia. Despite much progress over the last decades, the overall survival of patients with acute lymphoblastic leukemia (ALL) is about 40 percent for adults and about 80 percent for children (Pui et al., Faderl et al., Larson and Stock). When pediatric ALL relapses, 50-95% of children will not be cured (Gaynon et al.). There is a need in the art for effective treatments for hematological diseases and solid tumors. Herein, the inventors evaluate the role of ITGA6 in a new BCR-ABL1⁺ ITGA6^{fl/fl} mouse model (B220+CD19+) and in primary ALL and identify integrin ITGA6-specific targeting of ALL as a novel therapy to eradicate leukemia.

### SUMMARY OF THE INVENTION

The following embodiments and aspects thereof are described and illustrated in conjunction with systems, compositions and methods which are meant to be exemplary and illustrative, not limiting in scope.

Provided herein is an integrin alpha 6 specific monoclonal antibody. In one embodiment, the monoclonal antibody is P5G10 or a fragment, derivative or variant thereof. In another embodiment, the monoclonal antibody is P1H8 or a fragment, derivative or variant thereof.

Also provided herein are methods for treating, inhibiting, reducing the severity of and/or preventing or reducing the likelihood of relapse of hematological malignancies in a subject in need thereof. The methods include administering to the subject an effective amount of a pharmaceutical composition comprising an inhibitor of integrin alpha 6. In some embodiments, the inhibitors are monoclonal antibodies that bind to integrin alpha 6. In various embodiments, the method further comprises administering chemotherapeutic agents, tyrosine kinase inhibitors, inhibitors of other adhesion molecules (for example, inhibitors of integrin alpha 4) or a combination thereof.

Further provided herein are methods for treating, inhibiting, reducing the severity of and/or preventing or reducing the likelihood of relapse of solid tumors in a subject in need thereof. The methods include administering to the subject an effective amount of a pharmaceutical composition comprising an inhibitor of integrin alpha 6. In some embodiments, the inhibitors are monoclonal antibodies that bind to integrin alpha 6. In various embodiments, the method further comprises administering chemotherapeutic agents, tyrosine kinase inhibitors, inhibitors of other adhesion molecules (for example, inhibitors of integrin alpha 4) or a combination thereof.

Also provided herein are methods for treating, inhibiting, reducing the severity of and/or preventing or reducing the likelihood of relapse of disease-states associated with overexpression of integrin alpha 6 in a subject in need thereof. The methods include administering to the subject an effective amount of a pharmaceutical composition comprising an inhibitor of integrin alpha 6. In some embodiments, the inhibitors are monoclonal antibodies that bind to integrin alpha 6. In various embodiments, the method further comprises administering chemotherapeutic agents, tyrosine kinase inhibitors, inhibitors of other adhesion molecules (for example, inhibitors of integrin alpha 4) or a combination thereof.

Also provided herein are pharmaceutical compositions and kits that include an inhibitor of integrin alpha 6. In some embodiments, the inhibitors are monoclonal antibodies that bind to integrin alpha 6. In one embodiment, the monoclonal antibody is P5G10 or a fragment, derivative or variant there.

### BRIEF DESCRIPTION OF FIGURES

Exemplary embodiments are illustrated in the referenced figures. It is intended that the embodiments and figures disclosed herein are to be considered illustrative rather than restrictive.

FIG. 1 depicts, in accordance with various embodiments of the present invention, that Integrin ITGA6 is expressed highly in primary pre-B ALL. A, Two gene expression data sets from groups of James Downing (Ross et al.) and Jacques van Dongen (van Zelm et al.) were re-analyzed. The different expression of ITGA6 between ALL patients and normal pre-B donor cells was represented by heatmap. B, Clinical information of primary pre-B ALL samples and their expression of human ITGA6 determined by FACS. C, Representative dot plots for ITGA6 expression of pre-B ALL and normal pre-B samples.

FIG. 2 depicts, in accordance with various embodiments of the present invention, that high expression levels of ITGA6 at the time of diagnosis is predict positive MRD on day 29. A,B, Two probesets of ITGA6 in MRD^{|} and MRD⁻ ALL cases of the COG P9906 study were analyzed. MRD was measured by flow cytometry at the end of induction therapy (day 29). P-values were obtained from Wilcoxon test for each probeset.

FIG. 3 depicts, in accordance with various embodiments of the present invention that ITGA6 deletion induces de-adhesion of BCR-ABL1^{|} ALL cells. A, Schematic for mouse leukemia model with inducible ITGA6 deletion (left panel) and phenotype of BCR-ABL 1(p210)⁻ ALL (right panel). B, Deletion of ITGA6 induced by Tamoxifen (1.5 μM) was confirmed at 144 hours post Tamoxifen incubation by flow cytometry. C, Deletion of ITGA6 was confirmed by PCR. D, Adherent CreER^{T2} and EmptyER^{T2} cells on mLaminin-coated plates (×400) (left panel) and percentage of adherence (right panel).

FIG. 4 depicts, in accordance with various embodiments of the present invention, that ITGA6 deletion induces apoptosis in BCR-ABL1⁺ ALL cells. A, Inducible deletion of ITGA6. B, Apoptosis analysis of CreER^{T2} and EmptyER^{T2} cells at various time points post-deletion AnnexinV and 7-AAD staining C, Cell cycle analysis of CreER^{T2} and EmptyER^{T2} cells at various time points post-deletion by BrdU flow cytometry. D, Western Blot of protein changes in CreER^{T2} and EmptyER^{T2} cells on Day3 and Day5 post-deletion.

FIG. 5 depicts, in accordance with various embodiments of the present invention, that ITGA6 deletion sensitizes ALL cells to Nilotinib. Viability of CreER^{T2} and EmptyER^{T2} ITGA6^{f/f} BCR-ABL1⁺ CreER^{T2} and EmptyER^{T2} cells were plated onto tissue culture plates (no further ligand coated) and treated with Tamoxifen (1.5 μM) and Nilotinib (0.02 μM or 0.2 μM) for 1 to 3 days. Cell viability was determined by trypan blue exclusion. Y axis indicates cell viability relative to initial.

FIG. 6 depicts, in accordance with various embodiments of the present invention, that the combination of in vivo deletion of ITGA6 with tyrosine-kinase inhibition eradicates leukemia cells. A, Bioluminescence imaging was performed at indicated days post-ALL cells injection. B, Kaplan-Meier survival curve was analyzed and MST for each group: EmptyER^{T2} (n=6) (MST=27 days), CreER^{T2} (n=5) (MST=54.5 days). EmptyER^{T2}+Nilotinib (n=6) (MST=39.5 days), CreER^{T2}+Nilotinib (n=5) (MST=undefined days as all mice are alive). *p=0.0001, Log-rank Test. C, D ITGA6 deletion was confirmed by FACS in BM cells of mice sacrificed at time of death of leukemia. E, Residual leukemia cells were determined in spleen and bone marrow by RT-PCR and F, PCR in the groups EmptyER^{T2}+Nilotinib and CreER^{T2}+Nilotinib.

FIG. 7 depicts, in accordance with various embodiments of the present invention, that integrin alpha6 blockade using anti-alpha6 mAb P5G10 de-adheres primary ALL. A, B: Four primary ALL cases, LAX7R (normal karyotype), ICN1, PDX2, TXL3 (BCR-ABL1⁺) were pre-incubated with purified anti-human α6 antibody (P5G10, grey bar) or its isotype control IgG1 (white bar) on plates coated with 50 μg/ml of human laminin-1 (Laminin-1) or fibronectin or PBS as control. Adhesion of ALL cells (×400) % was assessed. C: Viability of ALL cells (LAX7R, SFO2) treated with and without α6 blockade (P5G10) and VDL (LAX7R, left panel) or Nilotinib (0.02 μM) or DMSO control (SFO2, right panel) is depicted. D, E: LAX7R and SFO2 (F, G) were co-cultured long-term for 17 days with OP9 cells and treated with P5G10 (20 μM), control IgG1 alone or in combination with VDL (Vincristine, Dexamethasone, L-Asparaginase) (LAX7R) or Nilotinib (NTB) (SFO2). Cell viability was determined by trypan blue counts (D, F) or nnexin V/7AAD staining and flow cytometry (E, G).

FIG. 8 depicts, in accordance with various embodiments of the present invention, that ITGA6 blockade does not mobilize leukemia to the peripheral blood in vivo. Primary ALL cells, TXL3 (A-C) and PDX2 (D-F) (both BCR-ABL1⁺) and LAX7R (G-I) (BCR-ABL negative) were injected into NSG mice (2.5×10⁶ cells/mouse). After confirmation of leukemia cells in the peripheral blood of recipient mice, mice received 30 mg/kg P5G10 or PBS control. The % of human CD45+ and CD19+ in peripheral blood (PB) was analyzed by flow cytometry (A-B, D-E, G-H) before (pre), 1 and 3 days after (post) treatment (Tx). (C, F, I) Kaplan-Meier survival curve was analyzed and median survival time (MST) calculated for each leukemia case and group (n=3/group).

FIG. 9 depicts ITGA6 blockade sensitizes leukemia cells to chemotherapy and eradicates leukemia in vivo. (A) Treatment regimen (top panel) and bioluminescent imaging of mice transplanted with LAX7R cells (normal karyotype). NSG mice were treated with PBS (n=5), ITGA6 blocking Ab (P5G10) (n=5), VDL (n=5) or VDL+P5G10 (n=5) and imaged on Day 66 and Day 92 after leukemia cell transfer. A mouse with no leukemia injection treated only with luciferin at time of imaging was included as background control (Image Ctrl). (B) Kaplan-Meier survival curve was analyzed and MST was calculated for each group: PBS (MST=39 days), P5G10 (MST=31 days), VDL (MST=71 days), VDL+P5G10 (MST=185 days). (C) Flow cytometric analysis of human CD45 and CD19 of bone marrow (BM) or splenic cells (SPC) of animals sacrificed at Day 186 post-injection or found dead at day 185 post-injection.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4^{th} ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

For references on pediatrics, see Schwartz et al., The 5-Minute Pediatric Consult 4^{th} ed., Lippincott Williams & Wilkins, (Jun. 16, 2005); Robertson et al., The Harriet Lane Handbook: A Manual for Pediatric House Officers 17^{th} ed., Mosby (Jun. 24, 2005); and Hay et al., Current Diagnosis and Treatment in Pediatrics (Current Pediatrics Diagnosis & Treatment) 18^{th} ed., McGraw-Hill Medical (Sept. 25, 2006).

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Antibody” as used herein refers to polyclonal antibodies, monoclonal antibodies, humanized antibodies, single-chain antibodies, and fragments thereof such as Fab, F(ab′)2, Fv, and other fragments which retain the antigen binding function of the parent antibody. In various embodiments, the fragments of the antibodies may be produced by treating the antibodies described herein with proteases. For example, treating the monoclonal antibody with papain may yield three fragments, namely two Fab fragments and one Fc fragment. Alternately, treating the monoclonal antibody with pepsin may yield two fragments, namely F(ab′)₂ and Fc. In some embodiments, the fragments of the antibodies recognize and bind integrin alpha 6 so as to inhibit integrin alpha 6, similar to the full length antibody.

“Beneficial results” may include, but are in no way limited to, lessening or alleviating the severity of the disease condition, preventing the disease condition from worsening, curing the disease condition, preventing the disease condition from developing, lowering the chances of a patient developing the disease condition and prolonging a patient's life or life expectancy. In some embodiments, the disease condition is cancer. In some embodiments, the disease condition is an autoimmune disease.

“Chemotherapeutic drugs” or “chemotherapeutic agents” as used herein refer to drugs used to treat cancer including but not limited to Albumin-bound paclitaxel (nab-paclitaxel), Actinomycin, Alitretinoin, All-trans retinoic acid, Azacitidine, Azathioprine, Bevacizumab, Bexatotene, Bleomycin, Bortezomib, Carboplatin, Capecitabine, Cetuximab, Cisplatin, Chlorambucil, Cyclophosphamide, Cytarabine, Daunorubicin, Dexamathasone, Docetaxel, Doxifluridine, Doxorubicin, Epirubicin, Epothilone, Erlotinib, Etoposide, Fluorouracil, Gefitinib, Gemcitabine, Hydroxyurea, Idarubicin, Imatinib, Ipilimumab, Irinotecan, L-Asparaginase, Mechlorethamine, Melphalan, Mercaptopurine, Methotrexate, Mitoxantrone, Ocrelizumab, Ofatumumab, Oxaliplatin, Paclitaxel, Panitumab, Pemetrexed, Rituximab, Tafluposide, Teniposide, Tioguanine, Topotecan, Tretinoin, Valrubicin, Vemurafenib, Vinblastine, Vincristine, Vindesine, Vinorelbine, Vorinostat, Romidepsin, 5-fluorouracil (5-FU), 6-mercaptopurine (6-MP), Cladribine, Clofarabine, Floxuridine, Fludarabine, Pentostatin, Mitomycin, ixabepilone, Estramustine, or a combination thereof.

“Humanized antibodies” as used herein means that at least a portion of the framework regions of an immunoglobulin are derived from human immunoglobulin sequences.

“Hematological diseases” or “hematological disorders” or “hematological malignancies” as used herein refers to diseases including but not limited to any one or more of leukemia, lymphoma, Chronic Myeloproliferative Disorders, Langerhans Cell Histiocytosis, Multiple Myeloma/Plasma Cell Neoplasm, Myelodysplastic Syndromes, Myelodysplastic/Myeloproliferative Neoplasms or a combination thereof. In various embodiments, leukemia is any one or more of Acute Lymphoblastic Leukemia (ALL), Acute Myeloid Leukemia (AML), Chronic Lymphocytic Leukemia (CLL), Chronic Myelogenous Leukemia (CML), Hairy Cell Leukemia (HCL) or a combination thereof. In various embodiments, lymphoma is any one or more of AIDS-Related Lymphoma, Cutaneous T-Cell Lymphoma, Hodgkin Lymphoma, Mycosis Fungoides, Non-Hodgkin Lymphoma, Primary Central Nervous System Lymphoma, Sézary Syndrome, T-Cell Lymphoma, Cutaneous, Waldenström Macroglobulinemia or a combination thereof.

“Solid Tumors” as used herein refers to neoplasms or lesions formed by an abnormal growth of body tissue cells other than blood, bone marrow or lymphatic cells. Solid tumors consist of an abnormal mass of cells which may stem from different tissue types such as liver, colon, breast or lung and may metastasize to other organs. Examples of solid tumors include but are not limited to Adrenocortical Tumors (Adenoma and Carcinoma), Carcinoma, Colorectal Carcinoma, Desmoid Tumors, Desmoplastic Small Round Cell Tumor, Endocrine Tumors, Ewing Sarcoma, Germ Cell Tumors (Solid Tumor), Hepatoblastoma, Hepatocellular Carcinoma, Melanoma, Neuroblastoma, Osteosarcoma, Retinoblastoma, Rhabdomyosarcoma, Soft Tissue Sarcomas Other Than Rhabdomyosarcoma and/or Wilms Tumor.

“Mammal” as used herein refers to any member of the class Mammalia, including, without limitation, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. In certain embodiments, the mammal is a human subject. The term does not denote a particular age or sex. Thus, adult and newborn subjects, as well as fetuses, whether male or female, are intended to be included within the scope of this term.

“Monoclonal antibody” as used herein refers to an antibody composition having a homogeneous antibody population. The term is not limited regarding the species or source of the antibody, nor is it intended to be limited by the manner in which it is made. The term encompasses whole immunoglobulins as well as fragments such as Fab, F(ab′)2, Fv, and others which retain the antigen binding function of the antibody. Monoclonal antibodies of any mammalian species can be used in this invention. In practice, however, the antibodies will typically be of rabbit or murine origin because of the availability of rabbit or murine cell lines for use in making the required hybrid cell lines or hybridomas to produce monoclonal antibodies.

“Subject” or “individual” or “animal” or “patient” or “mammal,” is meant any subject, particularly a mammalian subject, for whom diagnosis, prognosis, or therapy is desired. In some embodiments, the subject has a hematological malignancy. In some embodiments, the subject had a hematological malignancy at some point in the subject's lifetime. In various embodiments, the subject's hematological malignancy is in remission, is re-current or is non-recurrent.

“Single chain antibodies” as used herein refer to antibodies prepared by determining the binding domains (both heavy and light chains) of a binding antibody, and supplying a linking moiety which permits preservation of the binding function. This forms, in essence, a radically abbreviated antibody, having only that part of the variable domain necessary for binding to the antigen. Determination and construction of single chain antibodies are described in U.S. Pat. No. 4,946,778 to Ladner et al.

“Humanized antibodies” as used herein means that at least a portion of the framework regions of an immunoglobulin are derived from human immunoglobulin sequences.

“Treatment” and “treating,” as used herein refer to both therapeutic treatment and prophylactic or preventative measures, wherein the object is to prevent or slow down (lessen) the targeted pathologic condition, prevent the pathologic condition, pursue or obtain beneficial results, or lower the chances of the individual developing the condition even if the treatment is ultimately unsuccessful. Those in need of treatment include those already with the condition as well as those prone to have the condition or those in whom the condition is to be prevented. Examples of cancer treatment include, but are not limited to, active surveillance, observation, surgical intervention, chemotherapy, immunotherapy, radiation therapy (such as external beam radiation, stereotactic radiosurgery (gamma knife), and fractionated stereotactic radiotherapy (FSR)), focal therapy, systemic therapy, vaccine therapies, viral therapies, molecular targeted therapies, or a combination thereof.

“Therapeutic agents” as used herein refers to agents that are used to, for example, treat, inhibit, prevent, mitigate the effects of, reduce the severity of, reduce the likelihood of developing, slow the progression of and/or cure, a disease. Diseases targeted by the therapeutic agents include but are not limited to carcinomas, sarcomas, lymphomas, leukemia, germ cell tumors, blastomas, antigens expressed on various immune cells, and antigens expressed on cells associated with various hematologic diseases, autoimmune diseases, and/or inflammatory diseases. In addition, they include solid tumors, for example, breast cancer and glioblastoma and other tumors that express the target gene integrin alpha 6.

“Tyrosine kinase inhibitors,” as used herein, refer to molecules and pharmaceuticals, the administration of which to a subject result in the inhibition of tyrosine kinase, an enzyme that can transfer a phosphate group from ATP to a tyrosine residue in a protein. Examples of tyrosine kinases include but not limited to Apatinib, Cabozantinib, Canertinib, Crenolanib, Crizotinib, Dasatinib, Erlotinib, Foretinib, Fostamatinib, Ibrutinib, Idelalisib, Imatinib, Lapatinib, Linifanib, Motesanib, Mubritinib, Nilotinib, Nintedanib, Radotinib, Sorafenib, Sunitinib, Vatalanib, Vemurafenib or a combination thereof.

### Therapeutic Methods

Leukemia cells may find a safe haven in the bone marrow and resist thereby chemotherapy treatment leading to relapse of the disease. The inventors have identified a central anchor molecule of leukemia cells, integrin alpha 6, and shown that targeting this molecule using inhibitors of integrin alpha 6 can interrupt the binding of leukemia cells to the safe haven making them thus vulnerable to therapy. Inventors data using a genetic conditional knockout mouse model of integrin alpha6 supports the role of integrin alpha6 in (BCR-ABL1+) leukemia. Integrin alpha6 deletion in combination with tyrosine-kinase inhibition ablates leukemia in contrast to TKI only treatment. The invention is based, at least in part, on these finding. Therefore, integrin alpha 6 may be a novel therapeutic target for treatment of hematological malignancies.

Accordingly, described herein is a method for treating hematological malignancies and/or solid tumors in a subject in need thereof. The method includes providing a composition comprising an inhibitor of integrin alpha 6 and administering an effective amount of the composition to the subject so as to treat the hematological malignancies in the subject.

Also provided is a method for inhibiting hematological malignancies and/or solid tumors in a subject in need thereof. The method includes providing a composition comprising an inhibitor of integrin alpha 6 and administering an effective amount of the composition to the subject so as to inhibit the hematological malignancies in the subject.

Further provided is a method for reducing the severity of hematological malignancies and/or solid tumors in a subject in need thereof. The method includes providing a composition comprising an inhibitor of integrin alpha 6 and administering an effective amount of the composition to the subject so as to reduce the severity of hematological malignancies in the subject.

Also described herein is a method for preventing or reducing the likelihood of relapse of hematological malignancies and/or solid tumors in a subject in need thereof. The method includes providing a composition comprising an inhibitor of integrin alpha 6 and administering an effective amount of the composition to the subject so as to prevent or reduce the likelihood of relapse of hematological malignancies in the subject.

Also provided herein are methods for treating, inhibiting or reducing the severity of disease-states associated with over-expression of integrin alpha 6 in subjects in need thereof. The methods include providing a composition comprising an inhibitor of integrin alpha 6 and administering an effective amount of the composition to the subject so as to treat, inhibit or reduce the severity of disease-states associated with over-expression of integrin alpha 6.

In exemplary embodiments, hematological malignancies or solid tumors include but are not limited to any one or more of leukemia, lymphoma, Chronic Myeloproliferative Disorders, Langerhans Cell Histiocytosis, Multiple Myeloma/Plasma Cell Neoplasm, Myelodysplastic Syndromes, Myelodysplastic/Myeloproliferative Neoplasms or a combination thereof.

In some embodiments, leukemia is any one or more of Acute Lymphoblastic Leukemia (ALL), Acute Myeloid Leukemia (AML), Chronic Lymphocytic Leukemia (CLL), Chronic Myelogenous Leukemia (CML), Hairy Cell Leukemia (HCL) or a combination thereof.

In some embodiments, lymphoma is any one or more of AIDS-Related Lymphoma, Cutaneous T-Cell Lymphoma, Hodgkin Lymphoma, Mycosis Fungoides, Non-Hodgkin Lymphoma, Primary Central Nervous System Lymphoma, Sézary Syndrome, T-Cell Lymphoma, Cutaneous, Waldenström Macroglobulinemia or a combination thereof.

In some embodiments, the inhibitors of integrin alpha 6 include but are not limited to any one or more of peptides, proteins, small molecules, nucleic acids, oligonucleotides, antibodies or a combination thereof. In some embodiments, the nucleic acid inhibitor is a siRNA molecule that inhibits synthesis of integrin alpha 6 or an aptamer specific to integrin alpha 6.

In some embodiments, the inhibitor of integrin alpha 6 is an antibody that recognizes and binds integrin alpha 6. The antibody may be any one or more of a monoclonal antibody or a fragment thereof, a polyclonal antibody or a fragment thereof, chimeric antibodies, humanized antibodies and single chain antibody. In one embodiment, the antibody is a monoclonal antibody. In an embodiment, the monoclonal antibody is P5G10. In another embodiment, the monoclonal antibody is P1H8. The P5G10 and P1H8 antibodies are produced as described in Wayner E. and Hoffstrom B. (Development of monoclonal antibodies to integrin receptors. Methods Enzymol. 2007; 426:117-53) and is incorporated by reference in its entirety.

As described herein, in various embodiments, the methods described herein further comprise providing additional cancer treatments (simultaneously or sequentially). Additional cancer treatments include, but are not limited to, active surveillance, observation, surgical intervention, chemotherapy, immunotherapy, radiation therapy (such as external beam radiation, stereotactic radiosurgery (gamma knife), and fractionated stereotactic radiotherapy (FSR)), focal therapy, systemic therapy, vaccine therapies, viral therapies, molecular targeted therapies, or a combination thereof. In some embodiments, the inhibitors of integrin alpha 6 (for example, monoclonal antibodies) are conjugated to therapeutic agents to form, for example, antibody-protein toxin conjugates (Immunotoxins) including but not limited to Diphteria Toxin (DT) or Pseudomonas Exotoxin A (PE) or Ricin-like toxin (Fitzgerald et al, Cancer Research, Oct. 15, 2011, Vol 71:6300-6309), antibody-radionuclide conjugates, antibody-drug conjugates including but not limited to conjugation to monomethyl auristatin E (MMAE), monomethyl auristatin F (MMAE F), calicheamycin, maytanisoid DM4 (Fitzgerald et al, 2011; Teicher and Chari, Clin Cancer Res Oct. 15, 2011 Vol 17; 6389-6397).

In various embodiments of the methods described herein, the therapeutic composition comprises an inhibitor of integrin alpha 6 and a targeting element that targets markers on the surface of cancer cells. Markers on the surface of cancer cells that may be targeted by the targeting elements of the compositions described herein include but are not limited to 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EGFRVIII, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R α, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2 or vimentin. Other antigens specific for cancer will be apparent to those of skill in the art and may be used in connection with alternate embodiments of the invention.

### Therapeutic Compositions

The methods described herein include administering to the subject an effective amount of a composition that includes an inhibitor of integrin alpha 6. In one embodiment, the inhibitor is a monoclonal antibody or a fragment thereof. In an embodiment, the monoclonal antibody is P5G10 or a fragment thereof. In another embodiment, the monoclonal antibody is P1H8 or a fragment thereof. In some embodiments, the inhibitors of integrin alpha 6 (such as monoclonal antibodies or fragments thereof) are conjugated to additional therapeutic agents.

In various embodiments, the composition further comprises any one or more of a chemotherapeutic agent, tyrosine kinase inhibitor, inhibitor of integrin alpha 4, inhibitors of cell adhesion molecules, inhibitors that target the interaction of leukemia cells with the endothelia, stromal or osteoblastic niche of the microenvironment (such as other integrins, CXCR4, CD44, L-selectin, E-selectin, Paxillin) or a combination thereof. In some embodiments, the chemotherapeutic agent, the tyrosine kinase inhibitor, the integrin alpha 4 inhibitor, the inhibitors of cells adhesion molecules or the combination thereof are administered sequentially to the composition comprising the integrin alpha 6 specific monoclonal antibody (for example P5G10 or a fragment thereof). In some embodiments, the chemotherapeutic agent, the tyrosine kinase inhibitor, the integrin alpha 4 inhibitor, the inhibitors of cells adhesion molecules or the combination thereof are administered simultaneously with the composition comprising the integrin alpha 6 specific monoclonal antibody (for example P5G10 or a fragment thereof).

In some embodiments, chemotherapeutic agents may be selected from any one or more of cytotoxic antibiotics, antimetabolities, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: treosulfan, and trofosfamide; plant alkaloids: vinblastine, paclitaxel, docetaxol; DNA topoisomerase inhibitors: doxorubicin, epirubicin, etoposide, camptothecin, topotecan, irinotecan, teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. Re. 36,397); and NU1025 (Bowman et al.).

In various embodiments, tyrosine kinase inhibitors include but are not limited to any one or more of Apatinib, Cabozantinib, Canertinib, Crenolanib, Crizotinib, Dasatinib, Erlotinib, Foretinib, Fostamatinib, Ibrutinib, Idelalisib, Imatinib, Lapatinib, Linifanib, Motesanib, Mubritinib, Nilotinib, Nintedanib, Radotinib, Sorafenib, Sunitinib, Vatalanib, Vemurafenib or a combination thereof.

In some embodiments, examples of inhibitors of integrin alpha 4 may be administered in addition to the composition comprising the integrin alpha 6 inhibitor (for example, a monoclonal antibody specific to integrin alpha 6 such as P5G10 monoclonal antibody or a fragment thereof or P1H8 monoclonal antibody or a fragment thereof). Examples of inhibitors of integrin alpha 4 include but are not limited to natalizumab, BIO5192 or a combination thereof.

Additional therapies that may be administered in addition to the composition comprising the integrin alpha 6 inhibitor (for example, a monoclonal antibody specific to integrin alpha 6 such as P5G10 or a fragment thereof or P1H8 or a fragment thereof), including but not limited to radiation, immunotherapy and/or hormonal therapy.

In various embodiments, therapies include, for example, radiation therapy. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy.

For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In various embodiments, therapies include, for example, immunotherapy. Immunotherapy may comprise, for example, use of cancer vaccines and/or sensitized antigen presenting cells. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). In some embodiments, the antibodies described herein are conjugated to therapeutic agents to form, for example, antibody-protein toxin conjugates (Immunotoxins), antibody-radionuclide conjugates, antibody-drug conjugates (Teicher and Chari, Clin Cancer Res Oct. 15, 2011 Vol 17; 6389-6397). Immunotherapy can also focus on using the cytotoxic lymphocyte-recognized epitopes of cancer cell lines.

In various embodiments, therapies include, for example, hormonal therapy, Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

The duration and/or dose of treatment with the therapies described herein may vary according to the particular therapeutic agent. An appropriate treatment time for a particular therapeutic agent will be appreciated by the skilled artisan.

In various embodiments, the subject is a mammal (e.g., mouse, rat, primate, non-human mammal, domestic animal such as dog, cat, cow, horse), and is preferably a human.

In various embodiments, an effective amount of the compositions comprising inhibitor of integrin alpha 6 (for example, a monoclonal antibody specific to integrin alpha 6 such as P5G10) is any one or more of about 0.01 to 0.05 μg/kg/day, 0.05-0.1 μg/kg/day, 0.1 to 0.5 μg/kg/day, 0.5 to 5 μg/kg/day, 5 to 10 μg/kg/day, 10 to 20 μg/kg/day, 20 to 50 μg/kg/day, 50 to 100 μg/kg/day, 100 to 150 μg/kg/day, 150 to 200 μg/kg/day, 200 to 250 μg/kg/day, 250 to 300 μg/kg/day, 300 to 350 μg/kg/day, 350 to 400 μg/kg/day, 400 to 500 μg/kg/day, 500 to 600 μg/kg/day, 600 to 700 μg/kg/day, 700 to 800 μg/kg/day, 800 to 900 μg/kg/day, 900 to 1000 μg/kg/day, 0.01 to 0.05 mg/kg/day, 0.05-0.1 mg/kg/day, 0.1 to 0.5 mg/kg/day, 0.5 to 1 mg/kg/day, 1 to 5 mg/kg/day, 5 to 10 mg/kg/day, 10 to 15 mg/kg/day, 15 to 20 mg/kg/day, 20 to 50 mg/kg/day, 50 to 100 mg/kg/day, 100 to 200 mg/kg/day, 200 to 300 mg/kg/day, 300 to 400 mg/kg/day, 400 to 500 mg/kg/day, 500 to 600 mg/kg/day, 600 to 700 mg/kg/day, 700 to 800 mg/kg/day, 800 to 900 mg/kg/day, 900 to 1000 mg/kg/day or a combination thereof.

Typical dosages of an effective amount of the one or more composition can be in the ranges recommended by the manufacturer where known therapeutic compounds are used, and also as indicated to the skilled artisan by the in vitro responses or responses in animal models. Such dosages typically can be reduced by up to about an order of magnitude in concentration or amount without losing relevant biological activity. The actual dosage can depend upon the judgment of the physician, the condition of the patient, and the effectiveness of the therapeutic method based, for example, on the in vitro responsiveness of relevant cultured cells or histocultured tissue sample, such as biopsied malignant tumors, or the responses observed in the appropriate animal models. In various embodiments, the compositions of the invention comprising the retinoid agonist may be administered once a day (SID/QD), twice a day (BID), three times a day (TID), four times a day (QID), or more, so as to administer an effective amount of the retinoid agonist to the subject, where the effective amount is any one or more of the doses described herein.

### Pharmaceutical Compositions

In various embodiments, provided herein are pharmaceutical compositions including a pharmaceutically acceptable excipient along with a therapeutically effective amount of the integrin alpha 6 inhibitor (for example, a monoclonal antibody specific to integrin alpha 6 such as P5G10), so as to treat, inhibit, reduce the severity of and/or prevent relapse or reduce the likelihood of relapse of hematological malignancies in a subject.

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal, parenteral or enteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the parenteral route, the compositions may be in the form of solutions or suspensions for infusion or for injection. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Typically, the antibodies are administered by injection, either intravenously or intraperitoneally. Methods for these administrations are known to one skilled in the art.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, U.S.A.) (2000).

Before administration to patients, formulants may be added to the composition comprising the inhibitor of integrin alpha 6 (for example, a monoclonal antibody specific to integrin alpha 6 such as P5G10 or a fragment thereof or P1H8 or a fragment thereof). A liquid formulation may be preferred. For example, these formulants may include oils, polymers, vitamins, carbohydrates, amino acids, salts, buffers, albumin, surfactants, bulking agents or combinations thereof.

Carbohydrate formulants include sugar or sugar alcohols such as monosaccharides, disaccharides, or polysaccharides, or water soluble glucans. The saccharides or glucans can include fructose, dextrose, lactose, glucose, mannose, sorbose, xylose, maltose, sucrose, dextran, pullulan, dextrin, alpha and beta cyclodextrin, soluble starch, hydroxethyl starch and carboxymethylcellulose, or mixtures thereof. “Sugar alcohol” is defined as a C₄ to C₈ hydrocarbon having an —OH group and includes galactitol, inositol, mannitol, xylitol, sorbitol, glycerol, and arabitol. These sugars or sugar alcohols mentioned above may be used individually or in combination. There is no fixed limit to amount used as long as the sugar or sugar alcohol is soluble in the aqueous preparation. In one embodiment, the sugar or sugar alcohol concentration is between 1.0 w/v % and 7.0 w/v %, more preferable between 2.0 and 6.0 w/v %.

Amino acids formulants include levorotary (L) forms of carnitine, arginine, and betaine; however, other amino acids may be added.

In some embodiments, polymers as formulants include polyvinylpyrrolidone (PVP) with an average molecular weight between 2,000 and 3,000, or polyethylene glycol (PEG) with an average molecular weight between 3,000 and 5,000.

It is also preferred to use a buffer in the composition to minimize pH changes in the solution before lyophilization or after reconstitution. Most any physiological buffer may be used including but not limited to citrate, phosphate, succinate, and glutamate buffers or mixtures thereof. In some embodiments, the concentration is from 0.01 to 0.3 molar. Surfactants that can be added to the formulation are shown in EP Nos. 270,799 and 268,110.

Additionally, antibodies (for example, a monoclonal antibody specific to integrin alpha 6 such as P5G10 or a fragment thereof or P1H8 or a fragment thereof) can be chemically modified by covalent conjugation to a polymer to increase their circulating half-life, for example. Preferred polymers, and methods to attach them to peptides, are shown in U.S. Pat. Nos. 4,766,106; 4,179,337; 4,495,285; and 4,609,546 which are all hereby incorporated by reference in their entireties. Preferred polymers are polyoxyethylated polyols and polyethylene glycol (PEG). PEG is soluble in water at room temperature and in some embodiments, has an average molecular weight between 1000 and 40,000, between 2000 and 20,000, or between 3,000 and 12,000. In some embodiments, PEG has at least one hydroxy group, such as a terminal hydroxy group. The hydroxy group may be activated to react with a free amino group on the inhibitor. However, it will be understood that the type and amount of the reactive groups may be varied to achieve a covalently conjugated PEG/antibody of the present invention.

Water soluble polyoxyethylated polyols are also useful in the present invention. They include polyoxyethylated sorbitol, polyoxyethylated glucose, polyoxyethylated glycerol (POG), etc. POG is preferred. One reason is because the glycerol backbone of polyoxyethylated glycerol is the same backbone occurring naturally in, for example, animals and humans in mono-, di-, triglycerides. Therefore, this branching would not necessarily be seen as a foreign agent in the body. The POG has a molecular weight in the same range as PEG. The structure for POG is shown in Knauf et al., 1988, J. Bio. Chem. 263:15064-15070 and a discussion of POG/IL C 2 conjugates is found in U.S. Pat. No. 4,766,106, both of which are hereby incorporated by reference in their entireties.

Another drug delivery system for increasing circulatory half-life is the liposome. Methods of preparing liposome delivery systems are discussed in Gabizon et al., Cancer Research (1982) 42:4734; Cafiso, Biochem Biophys Acta (1981) 649:129; and Szoka, Ann Rev Biophys Eng (1980) 9:467. Other drug delivery systems are known in the art and are described in, e.g., Poznansky et al., Drug Delivery Systems (R. L. Juliano, ed., Oxford, N.Y. 1980), pp. 253-315; M. L. Poznansky, Pharm Revs (1984) 36:277.

After the liquid pharmaceutical composition is prepared, it may be lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is administered to subjects using those methods that are known to those skilled in the art.

The dosage and mode of administration will depend on the individual. Generally, the compositions are administered so that antibodies are given at a dose between 1 μg/kg and 20 mg/kg, between 20 μg/kg and 10 mg/kg, between 1 mg/kg and 7 mg/kg. In some embodiments, it is given as a bolus dose, to increase circulating levels by 10-20 fold and for 4-6 hours after the bolus dose. Continuous infusion may also be used after the bolus dose. If so, the antibodies may be infused at a dose between 5 μg/kg/minute and 20 μg/kg/minute, or between 7 μg/kg/minute and 15 μg/kg/minute.

### Kits

The invention also provides a kit to treat, inhibit, reduce the severity of and/or prevent or reduce the likelihood of relapse of hematological malignancies in a subject in need thereof. The kit comprises a composition comprising the inhibitor of integrin alpha 6 (for example, a monoclonal antibody specific to integrin alpha 6 such as P5G10) and instructions for use of the composition for treating, inhibiting, reducing the severity and/or prevent or reduce the likelihood of relapse of hematological malignancies in subjects in need thereof.

The kit is an assemblage of materials or components, including at least one of the compositions described herein. Thus, in some embodiments the kit contains a composition including an inhibitor of integrin alpha 6 (for example, a monoclonal antibody specific to integrin alpha 6 such as P5G10 or a fragment thereof or P1H8 or a fragment thereof).

The exact nature of the components configured in the inventive kit depends on its intended purpose. In one embodiment, the kit is configured particularly for human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to affect a desired outcome, such as so treat, inhibit, reduce the severity of and/or prevent or reduce the likelihood of relapse of hematological disorders in a subject. Optionally, the kit also contains other useful components, such as, measuring tools, diluents, buffers, pharmaceutically acceptable carriers, syringes or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a bottle used to contain suitable quantities of composition comprising the inhibitor of integrin alpha 6 (for example, a monoclonal antibody specific to integrin alpha 6 such as P5G10 or a fragment thereof or P1H8 or a fragment thereof). The packaging material generally has an external label which indicates the contents and/or purpose of the kit and/or its components.

### Production of Antibodies

In addition to the P5G10 antibody produced by the methods described in Wayner E. and Hoffstrom B. ((Development of monoclonal antibodies to integrin receptors. Methods Enzymol. 2007; 426:117-53), any other monoclonal antibody specific to integrin alpha 6 (such as P1H8) may be used with the methods described herein. Methods for producing such antibodies will be apparent to a person of skill in the art.

Antibodies that specifically recognize and bind integrin alpha 6 may be produced by any one of several methods known in the art. For example, see Yoshida et al., Experientia 43:329, 1987; Yoshida and Ichiman, J. Clin. Microbiol. 20:461, 1984; and U.S. Pat. No. 5,770,208 D. Lane, Antibodies: A Laboratory Manual (Cold Spring Harbor Press, Cold Spring Harbor N.Y., 1988); Kohler and Milstein, (1976) Eur. J. Immunol. 6: 511; Queen et al. U.S. Pat. No. 5,585,089; Riechmann et al., Nature 332: 323 (1988); and Ausebel et al., Current Protocols in Molecular Biology, John Wiley & Sons, Inc., N.Y., 1989. Classically, antigen-specific antibodies are produced by immunizing a host animal with the antigen (for example, the antigens set forth herein) and later collecting the antibody-containing serum from the animal.

Any animal capable of producing antibodies in response to an antigen may be used in the invention. Commonly used animals include: mice, rats, horses, cows, goats, sheep, rabbits, cats, dogs, guinea pigs, chickens and humans. Host animals are immunized by injection with the antigen. Preferably, after the first immunization, the host animal receives one or more booster injections of antigen to augment antibody production and affinity. For immunization of humans, care should be taken to select the appropriate antigen, adjuvant, and/or carrier protein to avoid potential adverse reactions (e.g., granuloma formation with Freund's complete adjuvant; anaphylactic shock).

To enhance the immunologic response antigens are typically mixed with adjuvant before injecting into a host animal or human. Adjuvants useful in augmenting antibody production include Freund's adjuvant (complete and incomplete), mineral gels such as aluminum hydroxide, surface active substances such as lysolecithin, pluronic polyols, polyanions, peptides, oil emulsions, KLH, and dinitrophenol (DNP). Examples of potentially useful human adjuvants include BCG (bacille Calmette-Guerin) and Corynebacterium parvum. Antigens can also be cross-linked or incorporated into lipid vesicles to enhance their antigenicity.

Antibodies within the invention include without limitation polyclonal antibodies, monoclonal antibodies, humanized, and chimeric antibodies. Polyclonal antibodies can be isolated by collecting sera from immunized host animals. Monoclonal antibodies can be prepared using the antigens discussed above and standard hybridoma technology. See, e.g., Kohler et al., Nature, 256:495, 1975; Kohler et al., Eur. J. Immunol, 6:511, 1976; Kohler et al., Eur. J. Immunol, 6:292, 1976; Hammerling et al., “Monoclonal Antibodies and T cell Hybridomas,” Elsevier, N.Y., 1981; Coligan et al., Current Protocols in Immunology, John Wiley & Sons, N.Y., 1997. Human monoclonal antibodies are prepared by immortalizing a human antibody secreting cell (e.g., a human plasma cell). See, e.g., U.S. Pat. No. 4,634,664. To obtain monoclonal antibodies, hybridomas or other immortalized antibody secreting cells are cultivated in vitro (e.g., in tissue culture) or in vivo (e.g., in athymic or SCID mice). Antibodies are isolated by collecting the in vitro culture medium or bodily fluids (e.g., serum or ascites) from the in vivo cultures.

Additionally, chimeric antibodies, which are antigen-binding molecules having different portions derived from different animal species (e.g., variable region of a rat immunoglobulin fused to the constant region of a human immunoglobulin), are expected to be useful in the invention. Such chimeric antibodies can be prepared by methods known in the art. E.g., Morrison et al., Proc. Nat'l. Acad. Sci. U.S.A., 81:6851, 1984; Neuberger et al., Nature, 312:604, 1984; Takeda et al., Nature, 314:452, 1984. Similarly, antibodies can be humanized by methods known in the art. For example, monoclonal antibodies with a desired binding specificity can be commercially humanized (Scotgene, Scotland; Oxford Molecular, Palo Alto, Calif.) or as described in U.S. Pat. Nos. 5,693,762; 5,530,101; or 5,585,089. In a like manner, portions of the constant region of Protein A- or Protein G-binding immunoglobulins can be altered, deleted or mutated to remove Protein A or Protein G reactivity.

Once isolated, antibodies can be further purified by conventional techniques including: salt cuts (e.g., saturated ammonium sulfate precipitation), cold alcohol fractionation (e.g., the Cohn-Oncley cold alcohol fractionation process), size exclusion chromatography, ion exchange chromatography, immunoaffinity chromatography (e.g., chromatography beads coupled to anti-human immunoglobulin antibodies can be used to isolate human immunoglobulins) and antigen affinity chromatography. See, e.g., Coligan et al., supra. Conventional antibody purification techniques using Protein A and Protein G (e.g., Protein A or Protein G chromatography) may be utilized.

Standard techniques in immunology and protein chemistry can be used to analyze and manipulate the antibodies of the invention. For example, dialysis can be used to alter the medium in which the antibodies are dissolved. The antibodies may also be lyophilized for preservation. Antibodies can be tested for the ability to bind specific antigens using any one of several standard methods such as Western Blot, immunoprecipitation analysis, enzyme-linked immunosorbent assay (ELISA), and radioimmunoassay (RIA). See, e.g., Coligan et al., supra.

Monoclonal antibodies may be prepared using the method of Kohler and Milstein, Nature (1975) 256:495-96, or a modification thereof. Typically, a mouse or rat is immunized as described above. However, rather than bleeding the animal to extract serum, the spleen (and optionally several large lymph nodes) are removed and dissociated into single cells. If desired, the spleen cells may be screened (after removal of nonspecifically adherent cells) by applying a cell suspension to a plate or well coated with the protein antigen. B-cells expressing membrane-bound immunoglobulin specific for the antigen bind to the plate, and are not rinsed away with the rest of the suspension. Resulting B-cells, or all dissociated spleen cells, are then induced to fuse with myeloma cells to form hybridomas, and are cultured in a selective medium (e.g., hypoxanthine, aminopterin, thymidine medium, “HAT”). The resulting hybridomas are plated by limiting dilution, and are assayed for the production of antibodies which bind specifically to the desired immunizing cell-surface antigen (and which do not bind to unrelated antigens). The selected mAb-secreting hybridomas are then cultured either in vitro (e.g., in tissue culture bottles or hollow fiber reactors), or in vivo (as ascites in mice).

If desired, the antibodies (whether polyclonal or monoclonal) may be labeled using conventional techniques. Suitable labels include fluorophores, chromophores, radioactive atoms (particularly ³²P and ¹²⁵I), electron-dense reagents, enzymes, and ligands having specific binding partners. Enzymes are typically detected by their activity. For example, horseradish peroxidase is usually detected by its ability to convert 3,3′,5,5′-tetramethylbenzidine (TMB) to a blue pigment, quantifiable with a spectrophotometer. “Specific binding partner” refers to a protein capable of binding a ligand molecule with high specificity, as for example in the case of an antigen and a monoclonal antibody specific therefor. Other specific binding partners include biotin and avidin or streptavidin, IgG and protein A, and the numerous receptor-ligand couples known in the art. It should be understood that the above description is not meant to categorize the various labels into distinct classes, as the same label may serve in several different modes. For example, ¹²⁵I may serve as a radioactive label or as an electron-dense reagent. HRP may serve as enzyme or as antigen for a mAb. Further, one may combine various labels for desired effect. For example, mAbs and avidin also require labels in the practice of this invention: thus, one might label a mAb with biotin, and detect its presence with avidin labeled with ¹²⁵I, or with an anti-biotin mAb labeled with HRP. Other permutations and possibilities will be readily apparent to those of ordinary skill in the art, and are considered as equivalents within the scope of the instant invention.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Production of P5G10 and P1H8 Antibodies

### Cell-Based ELISA

### Materials

PC3 cells passage 6 or 7 (ATCC #CRL-1435) grown in RPMI 1640 supplemented with 10% FBS; 96-well TC plates (Falcon #3072); 0.02% gelatin (2% stock from Sigma, G-1393), dilute 1/100 in PBS for coating plates; PBS as wash buffer (detergents may not be added to this buffer unless extraction or permeabilization of the cells is desider); 2% formaldehyde (dilute 37% stock with PBS); Trypsin 0.25%/EDTA solution (Gibco #25200); Blocking solution: 5% nonfat dry milk, NFDM (Carnation) 2% normal goat serum, GS (Gibco #16210-064); HRP conjugated goat anti-mouse IgG (gamma-chain specific, Southern Biotechnologies, Birmingham, Ala. #1030-05); HRP-conjugated goat anti-mouse IgM (mu-chain specific, Southern Biotechnologies, #1020-05); Substrate for HRP (ABTS turns blue green in the presence of HRP, Kirkgaard and Perry #50-66-18); SBTI (Sigma T-9777) stock solution at 1 mg/ml; Plate reader with filter set for O.D. 405.

### Procedure

96-well plates were coated with gelatin for 1 to 2 h at room temperature or overnight at 4°. PC3 cells were trypsinized (0.025% trypsin/EDTA) for 5 to 10 min at 37° and washed two times in RPMI supplemented with 10% FBS or 0.5 mg/ml SBTI. PC3 cells were plated at 5×10³ cells per well in RPMI 1640 supplemented with 10% FBS, and incubate at 37° for 1 to 2 days until the wells were confluent, ensuring that the cells are not allowed to overgrow so that or they stay on the plates for the duration of the ELISA. The plates were washed three times with PBS and fix for 15 to 30 min at room temperature with 2% formaldehyde. The plates were washed three times with PBS and blocked for 30 min at room temperature with 5% NFDM/2% GS. The plates were washed once and the antisera diluted into the first well (A1) at 1:100 and doubling dilutions were performed across the plate (1:100, 1:200, 1:400, etc.). Each sera was run in duplicate (two rows) so that one row received HRP-conjugated anti-IgG and one row received HRP-conjugated anti-IgM. The plates were incubated with primary for 1 h, and washed three times with PBS. HRP-conjugated anti-IgG or anti-IgM was added at 1/2000 (Southern Biotech) per well in block and incubated at RT for 30 min with rocking The plates were washed three times with PBS and ABT was added. The plates were incubated for 10 to 15 min at RT and read on a plate reader at O.D. 405.

### Cell Adhesion Assay

Cells can be labeled before the assay with radioactive isotopes (Cr 51) or fluorescent dyes such as calcein AM (Molecular Probes) or after (crystal violet staining) the completion of the assay. There is an excellent protocol for using calcein labeled cells in an adhesion assay on the Invitrogen website (Handbook Section 15.6, “Probes for Cell Adhesion”). The objective of the protocol described here is to stain the adherent cells with crystal violet for use with a standard plate reader (O.D. 600 filter set).

### Materials

PC3 cells grown in 15-cm tissue culture dishes. Four 15-cm plates will be needed to screen 6×96-well plates in a single fusion; Blocking solution (PBS supplemented with 10 g/l heat denatured BSA (dissolve BSA in PBS and heat to 80° for 3 min and plunge into 4° water bath until completely cooled; do not overheat)); Adhesion buffer (RPMI 1640-HEPES supplemented with 10mg/ml HBSA (heat-denatured BSA); 48-well plates (TC or non-TC); Protein solutions containing purified ECM components (Chemican, Linscott Directorylisting) made up at 2 to 5 μg/ml in PBS or Voller's Buffer (0.05 M of bicarbonate at pH 9.6); (A higher pH may help solubilize the ECM proteins when diluted. It is advisable to run the purified proteins on a gel under reducing conditions to control for purity and protein concentration); 37% formaldehyde (10× stock); For the crystal violet stain, 0.5% crystal violet (Sigma C-3886) in Me0H (5× stock); 3N NaC1 (20× stock); Methanol.

### Procedure

The number of plates needed to screen the fusion was determined. Two 48-well plates (Falcon #3078) per fusion plate were used. 96-well plates were not used because the background adhesion may complicate interpretation of the results. Protein was diluted to 5 μg/ml with PBS or Voller's buffer (0.05 M bicarbonate, pH 9.6). 200 μl of protein solution was added per well. 10ml of protein solution per 48-well plate was needed. The plates were incubated at 4° for 24 h. Prior to initiating the cell adhesion assay, the protein-coated plates were washed three times with PBS and blocked with 0.5 ml PBS/HBSA per well for 30 min at room temperature. After one wash, 50 μl of adhesion buffer was added per well. While the plates were blocking, cells were released from tissue culture dishes (trypsin, EDTA, or scraping), and washed two times with adhesion buffer supplemented with 20 μg/ml SBTI if trypsin was used. Cells were pelleted after final wash and resuspended at 1×10⁶ per milliliter in adhesion buffer and kept at room temperature until used. 200 μl of hybridoma supernatants was added per well. Both, a positive (anti-β1) and a negative (no antibody) control well were included. 50 μl of cell suspension was added to each well containing 5×10⁴ cells per well and incubated with hybridoma supernatants on 48-well plates with rocking for 15 min at room temperature to pre-bind antibodies to the cell surface. After 15 min, plates were placed in the incubator and incubated for 30 min. After 30 min, plates were checked to ensure that adhesion was adequate and spread in the assay in the control wells. If the cells in the negative control wells were not adhered and/or spread, the assay was allowed to run incubate for another 15 to 30 min. Once the wells were washed with PBS, the adherent cells were fixed with 3.7% formaldehyde for at least 3 h or overnight. The formaldehyde was washed off with PBS at least four times; crystal violet stain was prepared (0.15 mM NaCl/0.1% crystal violet, 20% MeOH) and 200 μl of stain was added to each well for 10 min at room temperature. The stain was diluted in each well by adding 1.0 ml of tap water or by filling the wells to the brim. The stain was aspirated and the wells were refilled with tap water two more times. It is very important to remove any residual stain. The plates were read in the microscope and the presence of functional antibodies was determined. The plates were blot dried and air dried. The crystal violet in the adherent cells was released with 200 μl of MeOH per well and the contents of each well was transferred to a 96-well ELISA plate and read on a standard ELISA plate reader at O.D. 600.

If a functionally inhibitory antibody is obtained, the data are usually unequivocal and can be evaluated by simply looking at the wells in the microscope. Sometimes the wells with a function-blocking antibody were blank. Sometimes aggregated cells in sheets were observed, which often results from disruption of cell substrate adhesion triggering cell—cell adhesion (cell aggregation). Additionally, partial inhibition may be seen where about 50% of the cells remain. Often this may indicate the presence of a functional antibody, and cloning was performed. Once the antibody was cloned, its functional capabilities were more accurately characterized.

### Production of Monoclonal Antibodies via Cell Fusion

The first step before performing a fusion with an immune spleen was to ensure that the polyclonal antiserum contained the appropriate antibody repertoire. Once this was established, the spleen was prepared for fusion with myeloma cells by performing the final injection (Day −3) or series of injections (Days −3, −2, −1). On Day 0, the mouse was sacrificed and the spleen was removed. The spleen was quite large and showed evidence of germinal centers (white areas). If IFA had been used more than once, the spleen could be embedded in connective tissue in the abdomen. It was blunt dissected, with care being taken not to puncture the stomach or intestines. The spleen was removed and dipped in 70% ethanol before smashing on a screen.

### Materials

Immunized RBF/DnJ mouse (Jackson Laboratories, stock #000726); 3- to 4-week BALB/cByJ mice for thymocyte feeder cells (Jackson Laboratories, stock #001026); Sterile instruments; FOX-NY myeloma cells (available from this laboratory or the ATCC [CRL-1732]); FBS (fetal bovine serum, Hyclone Laboratories, Logan Utah); Serum-free RPMI-1640 (Gibco #11875) for washing the cells (Heat 30 ml to 37° for the fusion); AAT medium (RPMI-1640 media (Gibco #11875), 500 ml; 100× 1-glutamine solution (Gibco #25030), 5 ml; 100× penicillin-streptomycin solution (Gibco #15140), 5 ml; 100× sodium pyruvate (Gibco #11360); 1000× gentamicin solution (Sigma G-1522), 0.5 ml; 50× adenine/aminopterin/thymidine (AAT Sigma A-5539), 1 vial); 50% polyethylene glycol 1500 (PEG 1500, Roche #783 641); 100-mesh screens (Sigma S3895-5EA); CO₂ incubator set at 5% CO₂ in air; Glass beakers for preparing makeshift water baths for use in the culture hood (x3); 50-ml centrifuge tube; Centrifuge set at 400×g; Plastic pipettes (2- and 10-ml); 4 to 8 ×96-well plates; 50× vial adenine/thymidine (AT, Sigma A-7422) to wean the resulting hybridomas once they have been cloned (In general, however, the selection pressure may be maintained by growing the hybridomas in AAT media until they have been through several rounds of cloning.)

### Prepare the FOX-NY Myeloma Cells

Thaw FOX-NY cells and plate one-half into RPMI supplemented with 10% FBS and one-half into media supplemented with 1× AAT (see below). The purpose of this AAT test is to ensure that the myeloma cells have not reverted to an aminopterin-insensitive state. This will definitely happen if the FOX-NY cells are maintained for long periods of time in tissue culture. Thaw the FOX-NY cells only when they are needed. Store them in liquid nitrogen at all other times. We keep hundreds of vials in our LN2 bank that we have quality controlled for their ability to support fusion and antibody development. The unfused FOX-NY cells should all be dead in 3 to 4 days. If not, then you need to return to another earlier frozen seed or to your original stock. Culture FOX-NY cells to 5×10⁵ to 10⁶ per milliliter in RPMI 1640 plus 10% FBS. You will need 1×10⁸ FOX-NY cells in exponential growth for the fusion. We usually culture three T75 flasks with 50 to 75 ml of media for 3 days before fusion. Do not grow the FOX-NY in the presence of antibiotics. You want to know if these cells become contaminated. On Day 0, pour the FOX-NY cells into 50-ml tubes, centrifuge at 400×g, and pool into one tube and wash three times in RPMI-1640 (no FBS). Resuspend in 20 ml and count in a hemocytometer. You will need 1×10⁸ cells.

### Preparation of the Spleen for Fusion

On Day 3, scrape PC3 cells were scraped with a rubber policeman from three 15-cm tissue culture plates. The cells were washed three times and resuspended into a final volume of 0.5 ml and sucked into a 1-ml syringe with an 18-gauge needle. A 23-gauge needle was used to inject the cells intraperitoneally into the RBF mouse chosen for fusion—in this case, the 1L mouse. Adjuvant was not used. If purified protein is used, additional injections on Days −2 and −1 may be administered. If using a protein or peptide preparation, select RIBI may be selected for the final injection(s). At Day 0, the spleen was removed and placed on a sterile screen in a 6-cm Petri dish. The screen may be purchased from Sigma and sterilized by auto-claving. A 100-mesh screen (Sigma #5389505EA) may be used. The spleen was kept dry on wet ice until processed to remove the cells. To make a single cell suspension of splenocytes, 2 ml of RMPI 1640 (no serum) was added to the spleen in the plate. The spleen was smashed with the plunger of a sterile 10-ml syringe until there were no more red clumps visible. Some connective tissue may remain in the screen; it was discarded. The splenocytes were washed off the screen and Petri dish and into a 50-ml tube with serum-free RPMI-1640. Serum was not added as this can stabilize the cell membranes. The purpose of the fusion protocol is to destabilize the cell membranes. Centrifugation was carried out at 400×g for 5 min at room temperature, and washed once with RPMI 1640 (no FBS). The spleen was resuspended in 10 ml of RPMI (no FBS).

### Fusion

5 ml of PEG 1500 and 30 ml of serum-free RPMI 1640 (no FBS) was warmed to 37° and moved into the hood in makeshift water baths. The preparation of the spleen was synchronized with the preparation of the FOX-NY cells. FOX-NY cells were washed and resuspended in 20 ml of RPMI (no FBS). Spleen cells and FOX-NY cells were counted. For the fusion, 5×10⁷ to 10⁸ spleen cells were fused with 1×10⁸ FOX-NY cells so that the fusion ratio was at least 0.5:1.0 spleen cells to FOX-NY. For 5×10⁷ spleen cells, four 96-well plates were used, and for 10×10⁸ spleen cells, eight 96-well plates were used. More than 8 plates per fusion were not set out. At least 5×10⁷ spleen cells were needed for a good fusion. Once the fusion ratio was set and the PEG and RPMI were warmed to 37°, the protocol described in Oi and Herzenberg (1980 Immunoglobulin-producing hybrid cell lines. In “Selected Methods in Cellular Immunology” B. B. Mishell and S. M. Shiigi, eds., pp. 351-372. Academic Press, New York) was followed exactly as described with a mini-water bath setup in the sterile hood to maintain the temperature as close to 37° as possible during fusion. The spleen cells and the FOX-NY myeloma cells were mixed together and centrifuged in a 50-ml tube at 400×g for 10 min at room temperature. All supernatant from the pellet was removed by aspirating and the pellet was warmed to 37° in the makeshift water bath and kept at 37° for all further manipulations. Using a 2-ml plastic pipette, 1 ml of warm PEG 1500 (50% v/v in media) was added to the cell pellet with gentle stirring over a 1-min period. The tip of the pipette was used to stir the pellet and keep immersed in the water bath. Stirring was continued for 1 min. With the same pipette, 1 ml of warm serum-free RPMI was gradually added over a 1-min period with gentle stirring. With the same pipette, another 1 ml of warm media was added with gentle stirring. Then over the next 2 to 3 min, with the same pipette another 7 ml of warm media was added. This brought the volume in the 50-ml tube up to 10 ml. Centrifugation was carried out at 400×g for 10 min. A 10-ml plastic pipette was used to break up the cell pellet and plate in AAT media supplemented with 20% FBS. This was a departure from the published Oi and Herzenberg (1980) protocol. The fusion was re-fed at Days 3 and 7 and screened at Day 10. Thymocytes may be used as feeder cells during the fusion or later for cloning. If thymocytes are used, 3- to 4-week BALB/c mice are sacrificed and one thymus provides enough feeder cells for 4×96-well plates. Thymocytes can also be frozen at a ratio of 1 thymus per two freezer vials (freezing mixture is 90% FBS and 10% DMSO). Then one freezer vial was thawed per two 96-well plates.

By Day 3 colonies were visible in the bottoms of the wells (10 to 20 cells). If the plate ratio described herein is followed for the number of spleen cells, three to four colonies per well should be visible by Day 7. If each well is screened by a capture ELISA, in a good fusion every single well should be positive for a mouse IgG. On Day 10 or 11, the media was quite yellow and the colonies were visible to the naked eye when the plates are held up to the light. The fusion was screened for the criteria decided upon, and cloned directly from the master wells (96-well plates). These plates may be frozen or the cells can be transferred to multicluster tubes (Costar 4411) in freezing mixture (90% FBS and 10% DMSO) and frozen. Multicluster plates may be kept at −80° or frozen in LN2 vapor for longer-term storage.

### Cloning

Positive hybridoma culture wells were cloned via the following protocol. Two dilutions were made. The first was made so that the cells were diluted to 1×10⁴/ml. Next, 100 μl of this cell suspension was further diluted to 10 ml in RPMI 1X AAT supplemented with 20% FBS and plated at 100 μl/well in a 96-well plate at 1 cell/well. Thymocyte feeder cells may be added at 1×10⁵ per well (or, as we have done on occasion, use Hybridoma Cloning Factor [Bioveris Corporation #210001] at 10%). However, allowing cells to get hooked on a growth factor is not advisable. Other dilutions may be used (limit dilutions), but we have found that cloning at one cell per well falls within the range of most cell lines. Cloning as soon as possible is advisable, and if possible, directly from the original master well (96-well plate). Fusion plates can be frozen by aspirating the supernatant and the addition of 100 to 200 μl of freezing mixture directly to each well (90%FBS and 10% DMSO). The plates were then placed into a −80° freezer and can be kept up to 1 year.

### Isotyping

Hybridoma culture supernatants may be isotyped in one of two ways: (1) capture ELISA and reaction with HRP-conjugated specific secondaries (Southern Biotech), or (2) using a Mouse Monoclonal Antibody Isotyping Kit (Roche #1-493-027). The capture ELISA protocol is identical to the one that we described for cells, except that instead of cells on the bottom of the wells, a purified goat anti-mouse reagent is coated at 1 to 2 μg/ml. Southern Biotech were used since they exhibit low cross-reactivity and good batch-to-batch variation. When the capture reagent was coated at 1 to 2 μg/ml or at 1/1000, the wells were blocked and the hybridoma culture supernatants were added at 100 μl/well. The specific HRP-conjugated secondaries were then added at 1/2000 (Southern Biotech) per well in block for 30 min at RT. Then the plates were washed and the HRP-substrate ABTS was added at 100 μl/well. When the Roche isostrips were used, prozone effects (Hoffstrom and Wayner, 1994; Immunohistochemical techniques to study the extracellular matrix and its receptors. Methods Enzymol. 245, 316-347) were observed and the culture supernatant samples were diluted to at least 1:100 or even 1:1000.

### Immunoprecipitation for Integrin Identification

### Cell Preparation

Cells were cultured and harvested before confluency either by trypsinization, scraping, or by treating with 2 mM EDTA for no more than 10 min. The cells were washed two times with PBS (see recipes below). If trypsin was used, the first wash contained soybean trypsin inhibitor at 0.5 mg/ml. Cell density was adjusted to 5×10⁶/ml in PBS (cations).

### Biotinylation and Lysis of Cells

A stock solution of biotin at 10 mg/ml in dimethyl sulfoxide (DMSO, nonsulfonated form, Pierce #21343) or water (sulfonated form Pierce #21217) was prepared immediately before use. Stock biotin was added to the cell suspension to a final concentration of 1:1000 (100 μg/ml), mixed and incubated at room temperature for 60 min with rocking to prevent cells from settling. The cells were washed three times with PBS plus 100 mM of glycine to block free amines, and once with PBS alone. The cell pellet was resuspended in 100 μl of formaldehyde-fixed Staphylococcus aureus bacteria 10% suspension (Sigma #P7155) or Pansorbin (Calbio-chem #507858) directly from vial. Pipetting was used to resuspend cell suspension with S. aureus bacteria. 1.0 ml of extraction (lysis) buffer was added per 1×10⁷ cells. The lysis buffer contained 1% Triton X-100 (or NP-40) 1 mM of PMSF (200 mM stock in absolute ethanol), 1 mM of N-ethylmalemide (200 mM stock in absolute ethanol), and 10 μg/ml of SBTI as protease inhibitors. Cells were lysed for 30 to 45 min at 4° with occasional vortexing. Lysis buffer may contain divalent cations at 1 mM, especially when working with the α4 receptor. Cell lysate was clarified by centrifugation at 10,000×g for 30 min. This is an essential step to remove nuclei and cytoskeletal components. The ysates were frozen at −80° or precleared for immune precipitation.

### Preclearing of Lysates

500 μl of packed fetuin-agarose (Sigma Cat #F-3256) was added per milliliter of lysate in 1.7-ml microcentrifuge tubes, incubate at 4° for 1 h with rocking, and centrifuged at 10,000×g for 15 min at 4°. The supernatant was decanted and added to fresh fetuin-agarose and lysated was precleared again. The lysates were precleared in this fashion a total of three times. The last preclear can go overnight if necessary.

Preparation and storage of Protein A Agarose

1 g of lyophilized protein A agarose (Sigma Cat #P-1406) in 40 ml of PBS pH 7.4 with 0.02% azide overnight on a mixer at 4° (1 g swells to approximately 4 ml) was swelled. The gel was pelleted by centrifugation, supernatant was decanted and the gel was washed by resuspending bed to 50 ml with PBS/azide. The gel was washed three times. After final wash, the gel was resuspended in PBS/azide in a 10% (v/v) solution and stored in the dark at 4°.

### Coupling of Rabbit Anti-Mouse IgG to Protein A-Agarose

The reason for using rabbit anti-mouse IgG to capture the mouse monoclonal antibodies on protein A beads was that rabbit IgGs have a very high affinity for protein A (higher than mouse IgGs). Rabbit IgGs also out-compete bovine IgG for sites on protein A. Hybridoma culture supernatants routinely contain up to 10% FBS, which can in turn contain up to 200 μg/ml bovine IgG. Therefore, if protein A is used for direct IPs without the benefit of pre-binding the anti-mouse then there may not be sufficient binding of the mouse IgGs to the protein A beads to bind the protein to the beads. Furthermore, we do not recommend using protein G agarose since unless employing purified mouse antibody, culture supernatant is used, which most likely contains 10% FBS. Unless low-Ig FBS is used, most FBS batches contain 100 to 200 μg/ml of bovine IgG, which will outcompete the mouse IgG at a ratio of 10:1 to 50:1 (since most mouse hybridoma culture supernatant contains 2 to 10 μg/ml of specific antibody). Thus, if protein G is used, most likely there will not be enough mouse IgG on the beads to IP the protein of interest. For the best results with hybridoma culture supernatant, use rabbit anti-mouse serum or purified rabbit IgG to capture mouse IgGs to protein A agarose.

To calculate the amount of rabbit anti-mouse serum to use, 10 μl of serum or 2 μg of purified IgG to 10 μl of packed protein A beads was used. Then 10 μl of packed rabbit anti-mouse beads per IP reaction was used. Rabbit anti-mouse antibody (Zymed Laboratories Cat #61-6500) was solubilized in 2 ml of deionized water. The unsed IgG was aliquoted and stored at −80°. Protein A agarose and rabbit anti-mouse were mixed at 4° for 1 to 2 h. A ratio of 0.1 ml rabbit anti-mouse to 1 ml of 10% protein A beads was used. With purified rabbit anti-mouse serum, 2 μg per IP reaction was used, keeping the volumes the same (0.1 ml rabbit anti-mouse to 1 ml of 10% protein A beads). Coupling of rabbit anti-mouse to protein A beads was done while preclearing the cell lysates. Rabbit anti-mouse-coupled protein A-agarose was pelleted by centrifugation, supernatant was decanted and pellet was washed three times by bringing the total volume up to 10 bead volumes with 1× IP buffer (see below). After the final wash, the pellet was resuspended in 1× IP buffer such that a 50-μl aliquot contained 10 μl of packed gel (i.e., a 20% suspension). Rabbit anti-mouse protein A beads may also be used to preclear lysates.

### Coupling of Test Mouse Antibody-Containing Supernatants to Rabbit Anti-Mouse IgG Protein A-Agarose

To each 1.7-ml microcentrifuge tube, 0.45 ml of test supernatant, 0.05 ml of 10× IP buffer (see below), and 0.05 ml of 20% rabbit anti-mouse IgG-coupled protein A-agarose was added. T tubes were mixed with rocking for 2 h (can be overnight) at 4°. The test mouse IgG coupled rabbit anti-mouse protein A-agarose beads was pelleted by centrifugation (use microcentrifuge at highest setting for 3 to 5 min). The supernatant was aspirated leaving a final volume of 50 μl and wash with 1.0 of 1× IP buffer once.

### Immunoprecipitation of Cell Lysate

To each tube containing washed and pelleted IP beads, 100 μl of precleared lysate was added and final volume was brought to 0.5 ml by adding 1× IP buffer per IP tube. Lysates were incubated with beads on a rocker for at least 2 h at 4°. The beads were pelleted by centrifugation (use a microfuge set a maximum for 3 to 5 min), supernatant was aspirated leaving a final volume of 0.05 ml, and IPs were washed with buffer four times with vortexing to resuspend pellet after each wash. The original supernatants may be saved and rotated to recycle the lysates for IP with another and distinct antibody, or they can be used for sequential preclear analysis to deter-mine the identity of the test antibody. Generally, to prove that a particular antibody can preclear another antibody (i.e., they react with the same protein), it took three rounds of preclear to complete. After the final wash and pelleting, supernatant was aspirated to approximately 0.025 ml and resuspended in 0.025 ml of 2× sample buffer (reduced or nonreduced) for SDS-PAGE gel analysis.

Releasing Immunoprecipitated Material from Beads

The tubes from above were boiled for 5 min and the beads were vortexed and pelleted as described herein. Supernatant was transferred to a stacking gel for SDS-PAGE. For IPs 1.5-mm gels may be used so that the entire IP will fit in the well created by the comb. If a few beads are also transferred, they may not affect how the gel runs. We used Invitrogen SeeBlue Plus 2 molecular weight makers as they run well and transfer well. Western blot analysis was performed to detect biotinylated and immune-precipitated integrin receptors.

### Example 2

### Experimental Methods

### Patient Samples

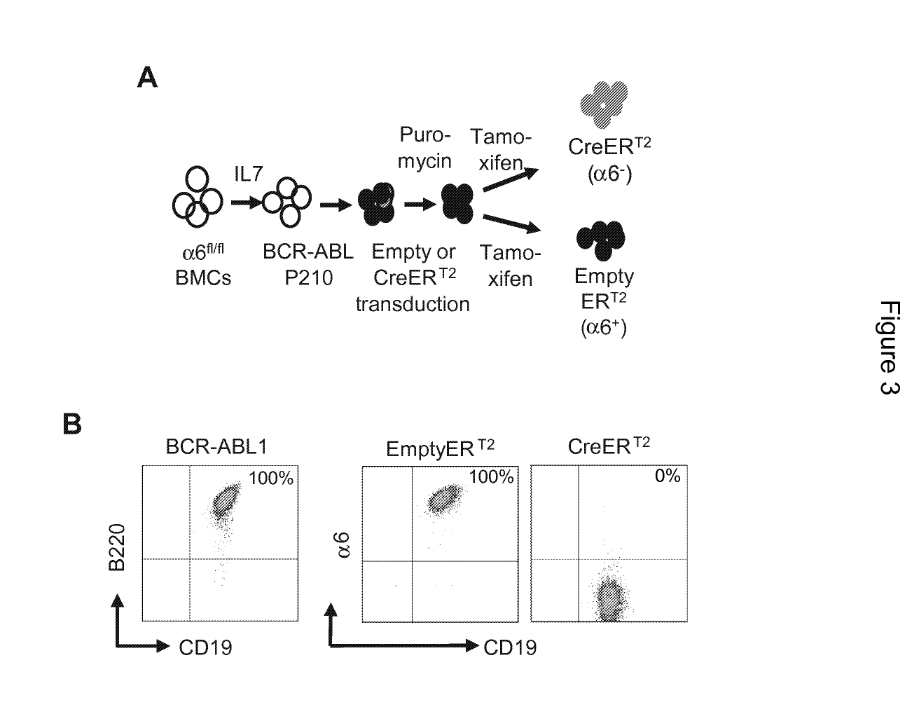

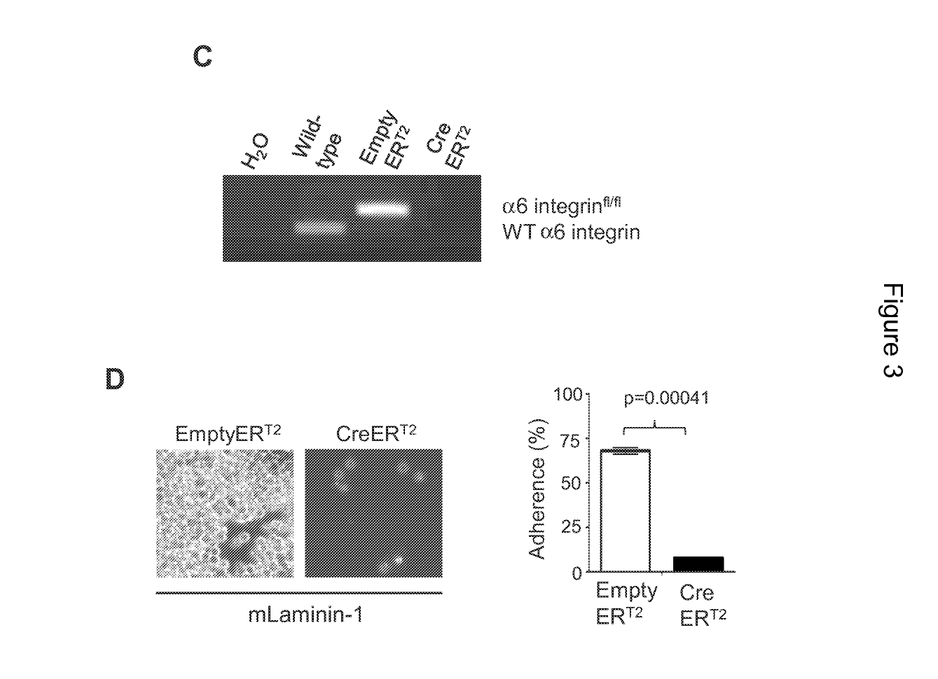

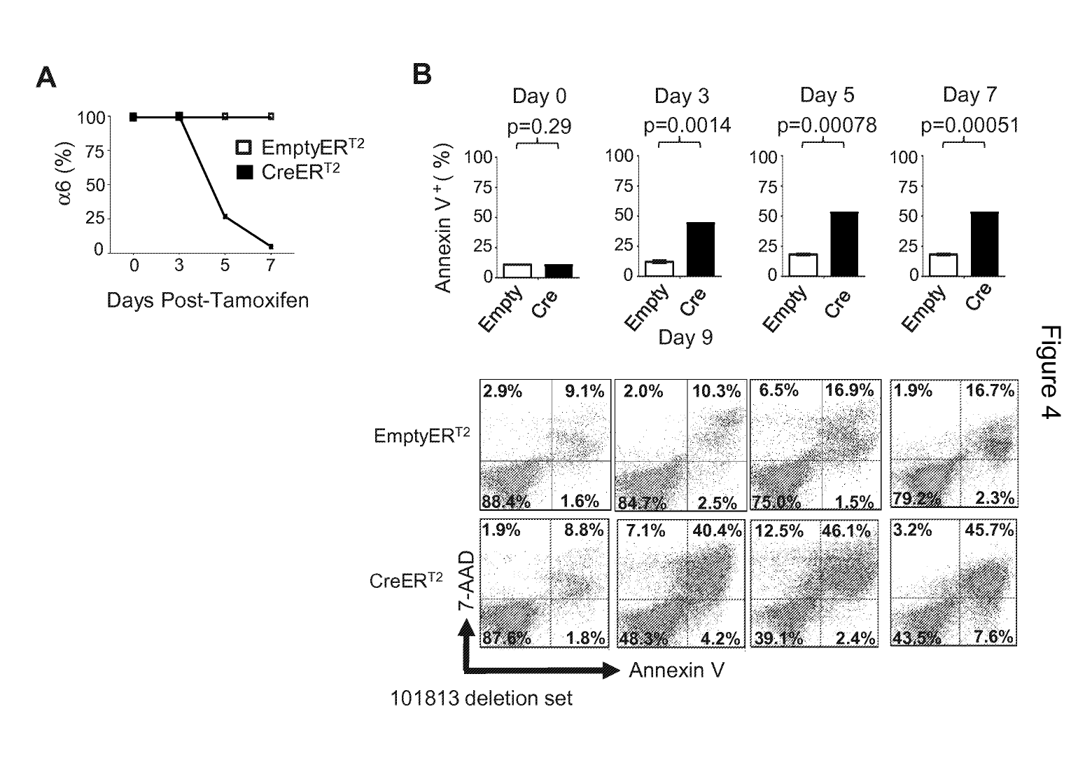

Bone marrow and peripheral blood samples from ALL patients (FIG. 2b) were acquired in compliance with the Institutional Review Board regulations of each institution. Informed consent for cell banking was obtained from all human subjects. Patient-derived (primary) leukemia cells were maintained in Roswell Park Memorial Institute Medium (RPMI-1640, Invitrogen, Carlsbad, Calif.) with GlutaMAX (Invitrogen) containing 20% fetal bovine serum (FBS) (Atlanta Biologica), 100 IU/ml penicillin and 100 μg/ml streptomycin (Invitrogen) at 37° C. in a humidified incubator with 5% CO2. Primary leukemia cells were cultured as described earlier (10, 11)) on murine OP-9 stroma cells with alpha-minimal essential medium (MEM) supplemented with 20% FBS, 100 IU/ml penicillin/100 μg/ml streptomycin.

### Microarray

Two gene expression datasets of microarray data of 60 ALL cases (15 patient cases per subtype of Pre-B ALL:E2A-PBX1, TEL-AML1, BCR-ABL, and MLL rearrangements) (http://www.stjuderesearch.org/data/ALL3) (12) and 15 normal pre-B cell cases (14 human cord blood CD34+lin− and precursor B cell subsets) (http://franklin.et.tudelft.nl)(13) were analyzed using BRB array tools for ITGA6 expression. The fold of ITGA6 overexpression was calculated by the ratio of the averaged signal intensity of 15 patient samples in each subtype of re-B ALL cases to the averaged 14 normal precursor B-cell cases. The calculated signal ratios of probesets were visualized as a heatmap with Java Treeview.

### Adhesion of Primary ALL Cells

Primary pre-B ALL cells were either pre-treated with anti-functional ITGA6 antibody (P5G10) or control Ab IgG2a (ebioscience) for 30 minutes at 37° C., washed once with PBS. Cells were then plated in triplicates onto human laminin-coated or murine OP9 stroma cells layered 24-well plates. After overnight incubation, suspension cells in the supernatant were removed by pipetting out of the supernatant media. The plates were washed once with 1 ml PBS. The adhering cells were photographed using a phase contrast Olympus IX71 microscope. The adhering cells were detached by pipetting up and down monitored by observation under microscope. The adhering cell count was determined by trypan blue exclusion of dead cells.

### In Vitro and In Vivo Model for BCR-ABL1-Transformed ITGA6fl/fl ALL and Bioluminescence Imaging

Bone marrow cells from ITGA6fl/fl mice (Georges-Labouesse et al.) mice were harvested and retrovirally transformed by BCR-ABL1 (Pear et al.) in the presence of 10 ng IL7 (Peprotech) in Retronectin-(Takara) coated plates. All BCR-ABL1-transformed ALL cells derived from bone marrow of mice were maintained in Iscove's modified Dulbecco's medium (IMDM) with GlutaMAX containing 20% fetal bovine serum (Atlanta Biologica), 100 IU/ml penicillin, 100 μg/ml streptomycin (Invitrogen), and 50 μM 2-mercaptoethanol (Sigma-Aldrich) at 37° C. in a humidified incubator with 5% CO2. After cytokine-independent proliferation, BCR-ABL1 transformed ITGA6fl/fl cells were transduced retrovirally with EmptyERT2 or CreERT2. For bioluminescent imaging, these cells were then transduced with a lentiviral vector (pCCL-MNDU3-LUC) encoding firefly luciferase and with a neomycin selection marker in 24 well plates coated with retronectin (Takara) for 48 hours as described previously (Park et al.). After 1 μg/ml puromycin selection, VLA6 deletion was induced in vitro by addition of 1.0 μM tamoxifen (Sigma-Aldrich). For in vivo deletion of ITGA6, 0.5×10⁶ luciferase-labeled ITGA6fl/fl BCR-ABL1+ EmptyERT2 or CreERT2 ALL cells were injected via tail vein into sublethally irradiated (350 cGy) NOD/SCID mice. ITGA6 in vivo deletion was induced by daily administration of 100 mg/kg˜150 mg/kg Tamoxifen per oral gavage on days 3-8 and 16-20 after leukemia cell transfer. Serial monitoring of leukemia progression in mice was performed as described earlier (Park et al) at indicated time points using an in vivo IVIS 100 bioluminescence/optical imaging system (Xenogen). D-Luciferin (Promega) dissolved in PBS was injected intraperitoneally at a dose of 2.5 mg per mouse 15 minutes before measuring the luminescence signal. General anesthesia was induced with 5% isoflurane and continued during the procedure with 2% isoflurane introduced via a nose cone. Mice were monitored for weight loss and other leukemia symptoms. Moribund mice were sacrificed and tissues were analyzed for leukemia cell infiltration to confirm leukemia as the cause of death. All mouse experiments were subject to institutional approval by Children's Hospital Los Angeles IACUC.

### Western Blotting

Cells were lysed in M-PER buffer (Thermoscientific) supplemented with a 1% protease inhibitor cocktail (Pierce) and proteins were separated by 4% to 12% SDS-PAGE and electro-transferred to PVDF membrane (Invitrogen). For the detection of mouse and human proteins by Western blot, primary antibodies were used together with the WesternBreeze immunodetection system (Invitrogen). Antibodies against β-actin were used as a loading control (AC-15, Santa Cruz Biotechnology). Blots were visualized using Alkaline Phosphatase-conjugated secondary antibody solution (Invitrogen) followed by Chemiluminescent substrate detection (Invitrogen) and exposed to Blue Lite Autorad film (GeneMate).

### PCR

Genomic DNA was extracted from BCR-ABL-1+ ITGA6fl/fl cells transduced with EmptyERT2 or CreERT2 at 6 days post-tamoxifen treatment (Qiagen Ltd.).

### Flow Cytometry

Annexin V and 7-AAD for apoptosis analyses were obtained from BD Biosciences. Antibodies included human and mouse CD45, CD56, CD3, CD49f and respective isotype control antibodies(IgG1, IgG2a).

Correlation of ITGA6 Gene Expression on Leukemic Blasts with Clinical Outcomes of Pre-B-ALL Patients

The patient clinical and gene expression microarray data were obtained from GEO database accession number GSE11877 of 207 high-risk B-precursor ALL patients from the Children's Oncology Group (COG) Clinical Trial P9906 (Kang et al). The patients were treated uniformly with a modified augmented Berlin-Frankfurt-Miinster Study Group (BFM) regimen and the patients with very high-risk features (BCR-ABL1 or hypodiploidy) were excluded in the study (Park et al). The majority of patients (n=191) had MRD assessed by flow cytometry; cases were defined as MRD positive or MRD negative at the end of induction therapy (day 29) using a threshold of 0.01%. The comparison of VLA6 expression in the MRD+ and MRD− patient groups were performed using Wilcoxon test in R package (R Development Core Team).

### In Vitro Drug Testing

For ITGA6 deleted and non-deleted murine cells, cells cultured on murine laminin or irradiated murine OP-9 were treated with VDL (0.5 nM Vincristine, 5 nM Dexamethasone, and 0.0005 IU L-Aparaginase). For human pre-B-ALL, cells were pre-treated with anti-functional anti-ITGA6 (CD49f) antibody P5G10 or IgG1 control antibody (eBioscience) combined with VDL or Nilotinib. Cell viability was determined by Trypan blue exclusion. Human ALL cell were blocked with purified anti-functional anti-ITGA6 Ab or control Ab (ebioscience) for 30 minutes and washed once with PBS. Cells were then plated in triplicates onto-human laminin-coated 24-well plates treated with either VDL (LAX7R) or imatinib (TXL3, ICN1) for 2 days.

### Isolation of NK Cells and Calcein-AM Release Assay

ADCC (Antibody-dependent cell-mediated cytotoxicity) of primary ALL cells was determined using a Calcein-AM release assay as previously described31. NK cells (>94% CD56+CD3−) from healthy donors were a gift from Dr. Nora Heisterkamp. Primary ALL cells were labeled with 5 μM Calcein-AM (Invitrogen) for 30 minutes at 37° C. and then treated with P5G10 or control Abs IgG1 and IgG4 and washed with PBS once. Primary ALL cells as target cells (T) were mixed with isolated NK cells as effectors cells (E) at E/T ratio from 5:1 to 15:1. After 1 hour incubation, cells were transferred to a black ViewPlate-96 plate and read on Filter Max F3 Microplate reader (Molecular devices, Sunnyvale, Calif.) using 485 nm excitation/535 nm emission filter set. % specific lysis was calculated by the following equation31: % specific lysis=(mean experimental release−mean spontaneous release)/(mean maximal release−mean spontaneous release)×100%.

### Example 3

Integrin ITGA6 Expression is Higher in Pre-B ALL than in Normal Pre-B Cells

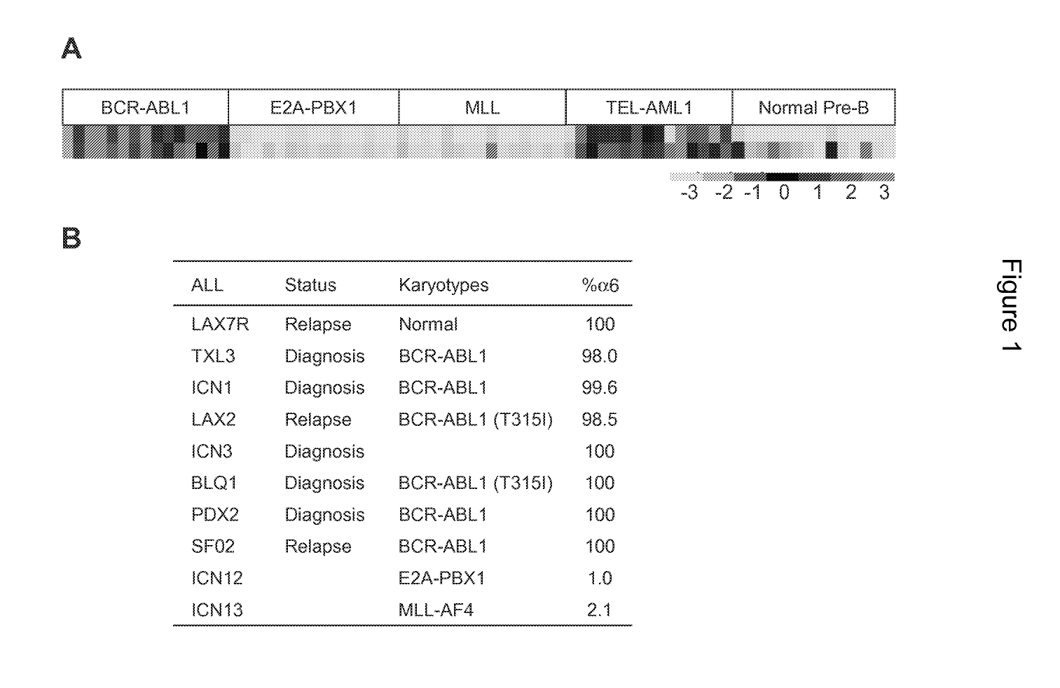

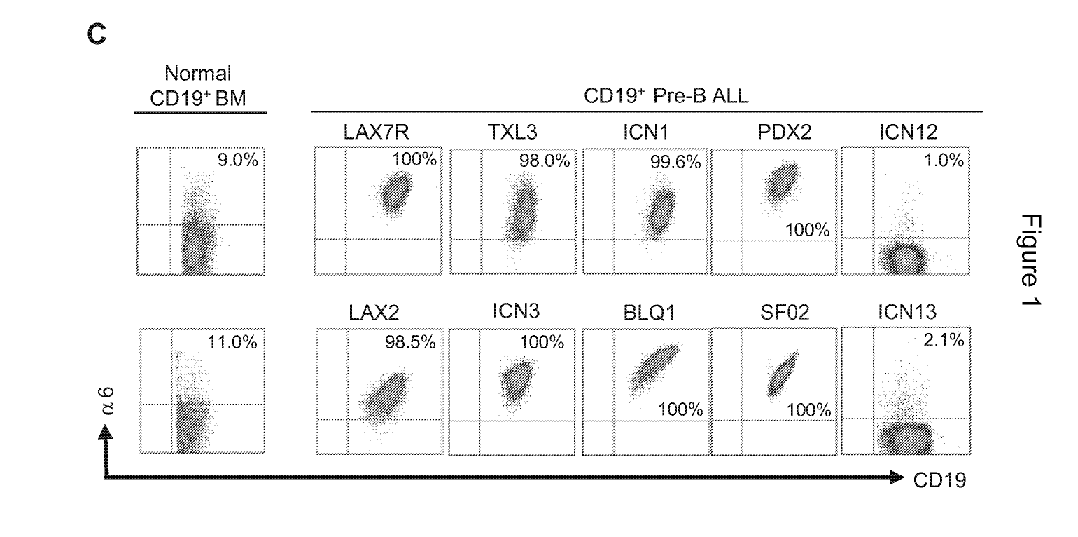

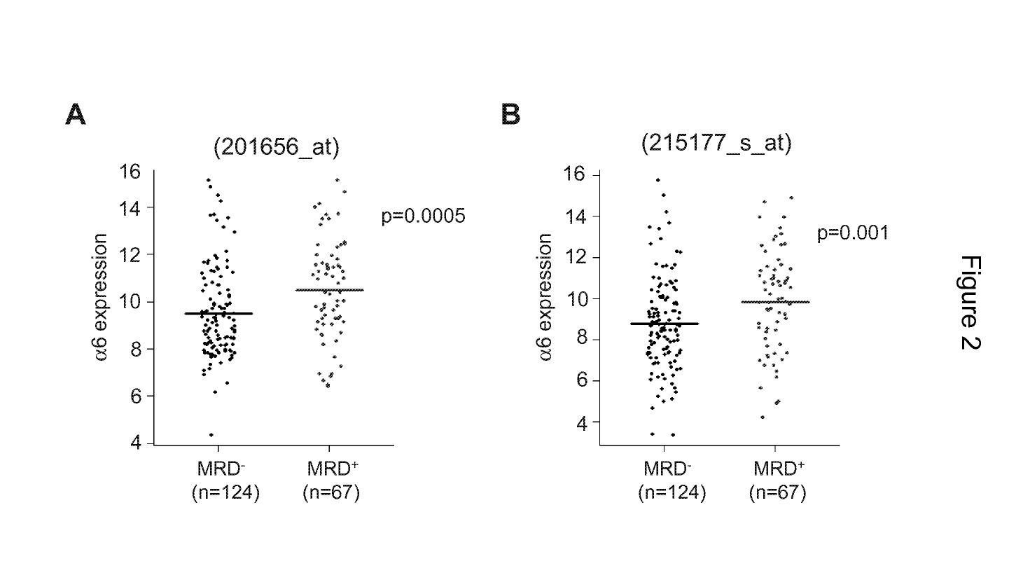

Two comparative gene expression datasets from 60 pre-B ALL patients (12) and 15 normal healthy donors (13) were first analyzed and showed marked overexpression of ITGA6 in BCR-ABL1-positive patients compared to normal pre-B cell cases (FIG. 1A). To verify this differential expression of ITGA6, patient-derived (primary) pre-B ALL cases were analyzed further for ITGA6 expression by flow cytometry (FIGS. 1B, C). ITGA6 was expressed in pre-B ALL and expression levels were significantly higher than CD 19+ pre-B cells of healthy bone marrow donors (FIG. 1C).

### Example 4

### High Expression Levels of ITGA6 at the Time of Diagnosis Predict Positive MRD on Day 29

To determine the significance of integrin ITGA6 in ALL patients, we correlated expression of ITGA6 with clinical outcome in 191 high-risk (BCR/ABL1−) pre-B ALL patients uniformly treated according to the Children's Oncology Group (COG) P9906 clinical trial (Kang et al.). The analysis revealed that high expression of ITGA6, the alpha chain of ITGA6, portends poor clinical outcomes in patients with ALL—patients with minimal residual disease (MRD+, measure on day 29 of induction) have a higher ITGA6 expression than the MRD− cases (p=0.0005 and p=0.001 respectively for two ITGA6 probesets) (FIGS. 2A, B).

### Example 5

### ITGA6 Deletion De-Adheres and Induces Apoptosis in BCR-ABL1+ Leukemia

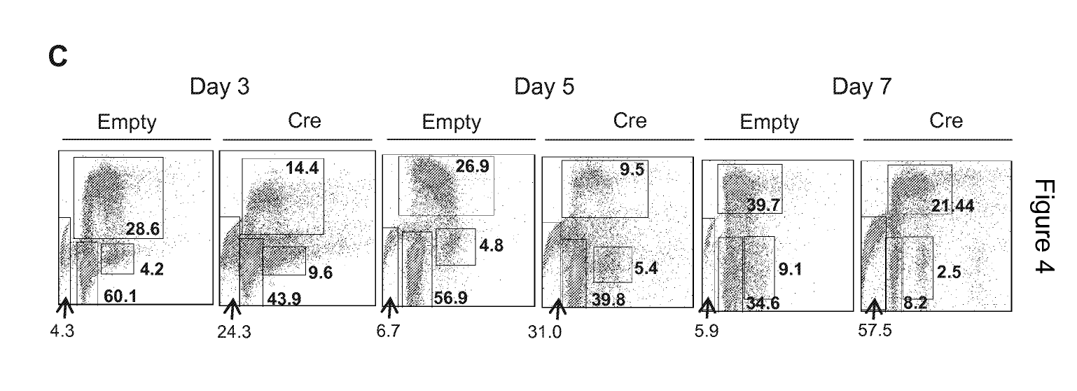

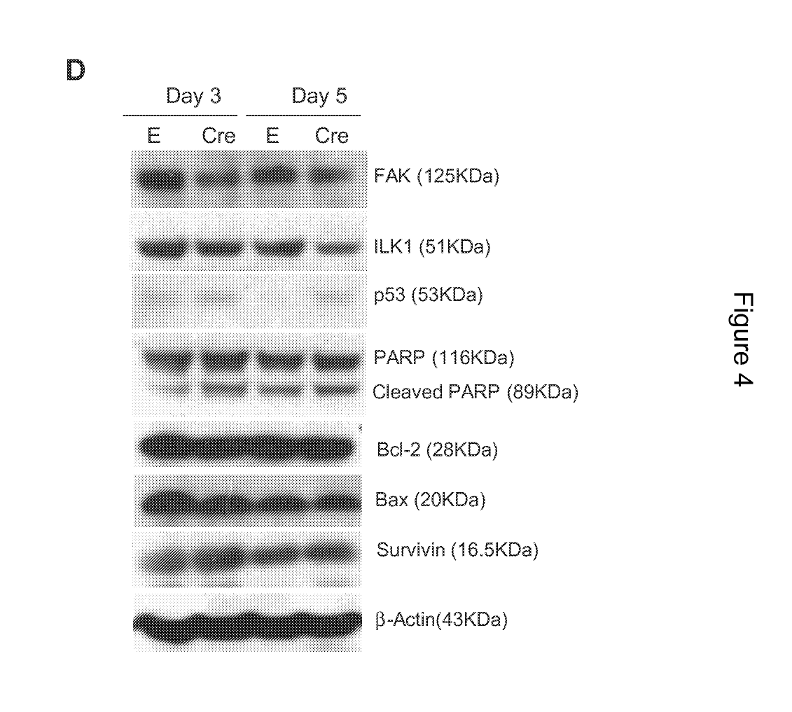

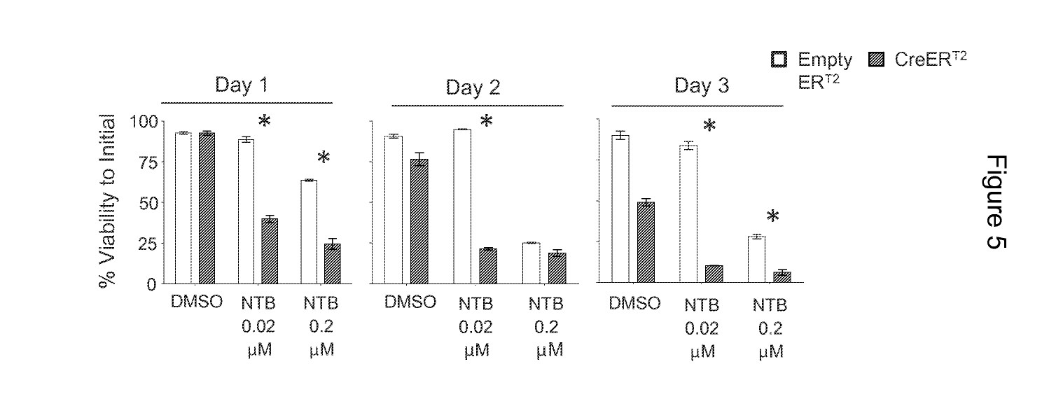

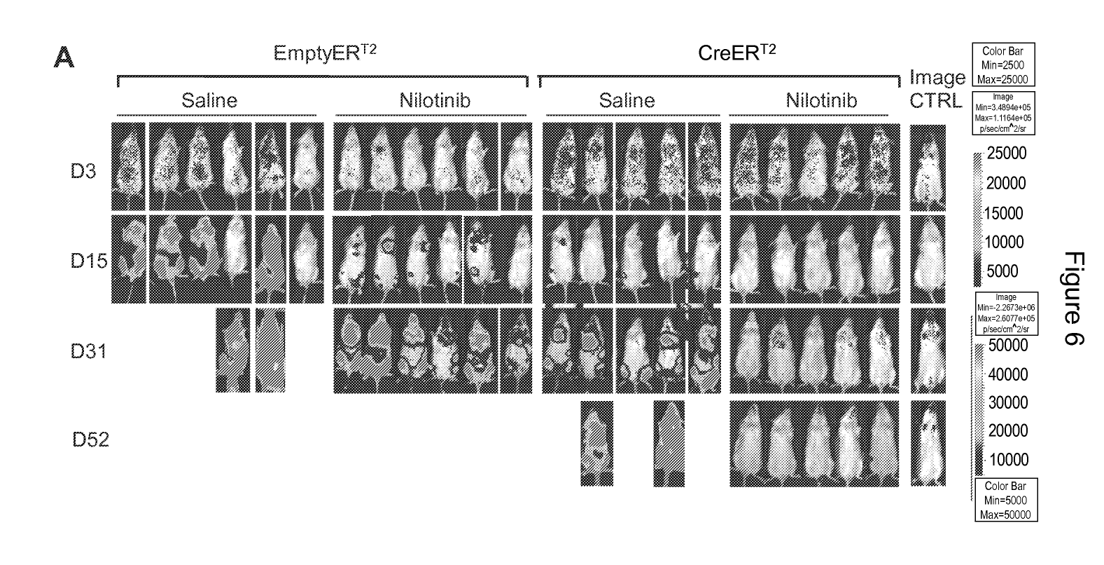

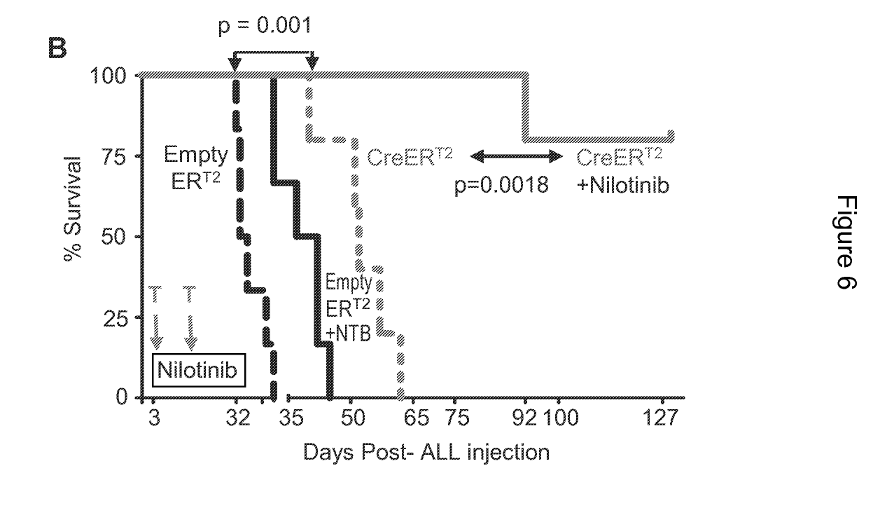

To study the function of these integrin receptors in a genetic experiment, we developed a BCR-ABL1+ mouse model (B220+CD19+) for loss of function of integrin ITGA6. ITGA6fl/fl cells were oncogenically transformed using BCR-ABL1 (p210) and cultured under lymphoid-skewing conditions (FIG. 3A). Induction of pre-B (B220+ CD19+) ALL was confirmed by flow cytometry (FIG. 3B, left panel). Subsequent transduction with CreERT2 or EmptyERT2 generated leukemia cells in which ITGA6 ablation could be induced (CreERT2) by in vitro addition of Tamoxifen (FIG. 3B). Conditional ablation of integrin ITGA6 in vitro was determined by flow cytometry (FIG. 3B, right panel) and PCR (FIG. 3C). ITGA6 deletion decreased adhesion significantly compared to undeleted controls (7.6%±0.08% vs. 68%±1.8%; p<0.05) by day 6 post-Tamoxifen treatment (FIG. 3D). Of note, the inducible deletion with Tamoxifen is a process of several days (FIG. 4A). We observed increased apoptosis of ITGA6-deleted leukemia cells as determined by analysis of AnnexinV and 7-AAD by flow cytometry (FIG. 4B) and cell cycle analysis by BrDU flow cytometry (FIG. 4C). Western Blotting of the ITGA6 deleted cells showed compared to ITGA6 competent cells decrease in total FAK, ILK1 on Day3 and Day5 post-Tamoxifen treatment as well as a decrease in anti-apoptotic gene BCL2 and survivin Day5 post-Tamoxifen treatment (FIG. 4D).

### Example 6

### ITGA6 Deletion Sensitizes Murine Leukemia to Tyrosine Kinase Inhibition

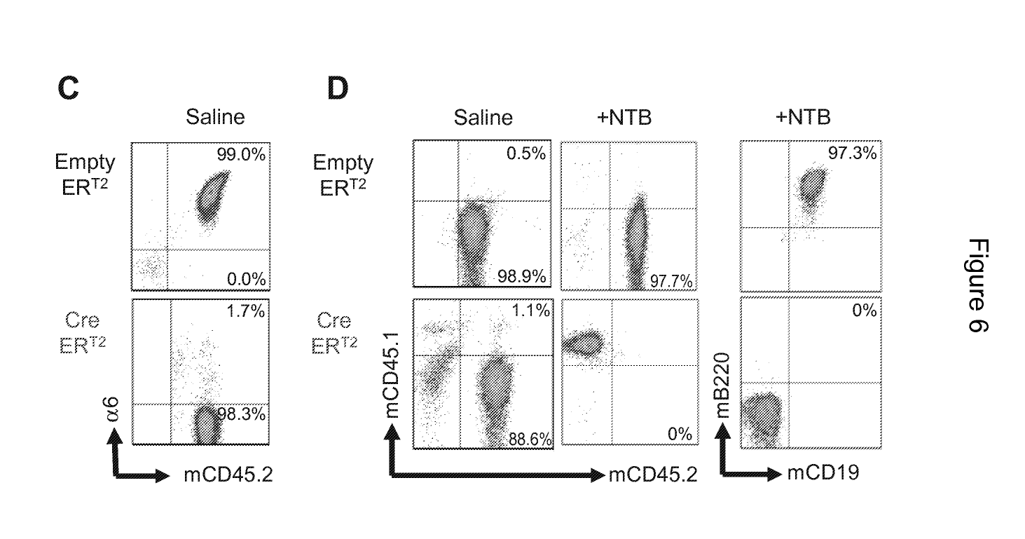

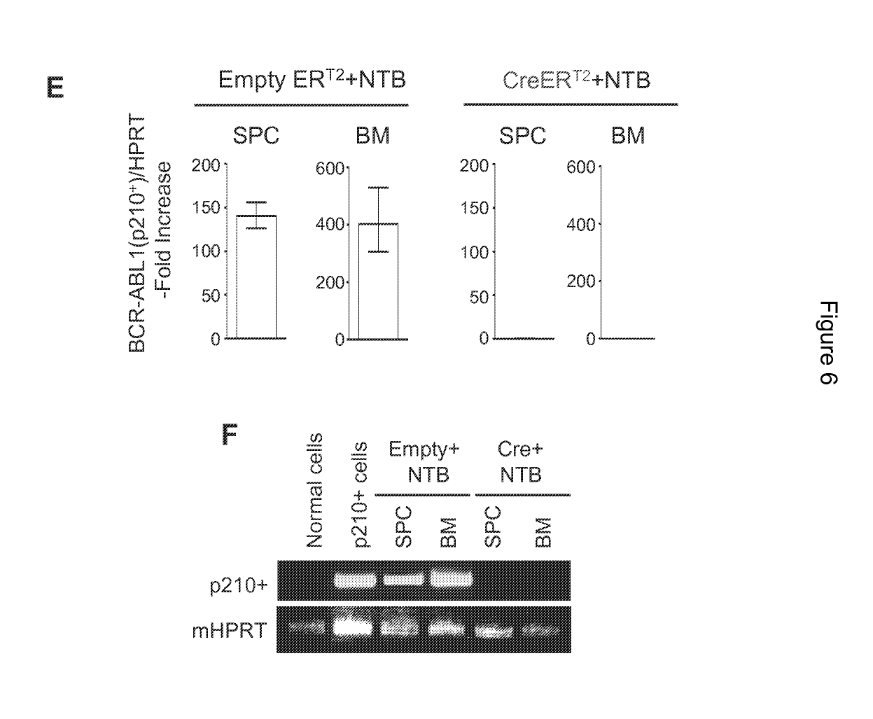

To determine the role of ITGA6 in chemoresistance, BCR-ABL-1+ ITGA6fl/fl cells transduced with EmptyERT2 or CreERT2 were treated with Tamoxifen for induction of ITGA6 deletion and at the same time with tyrosine kinase inhibitor Nilotinib (0.02 and 0.2 μM) or dissolvent DMSO (0.1%) as control. ITGA6 deletion markedly decreased drug resistance of CreERT2 cells to Nilotinib compared to EmptyERT2 cells, as demonstrated by the viability of leukemia cells on Day 3 post-treatment with Nilotinib (0.02 μM) (10.2%±0.1% vs. 83.7%±2.5%, p<0.05) (FIG. 5) indicating that ITGA6 deletion sensitizes murine leukemia to tyrosine kinase inhibition.

### Example 7

Combination of ITGA6 Deletion with Tyrosine Kinase Inhibition Eradicates Murine Leukemia

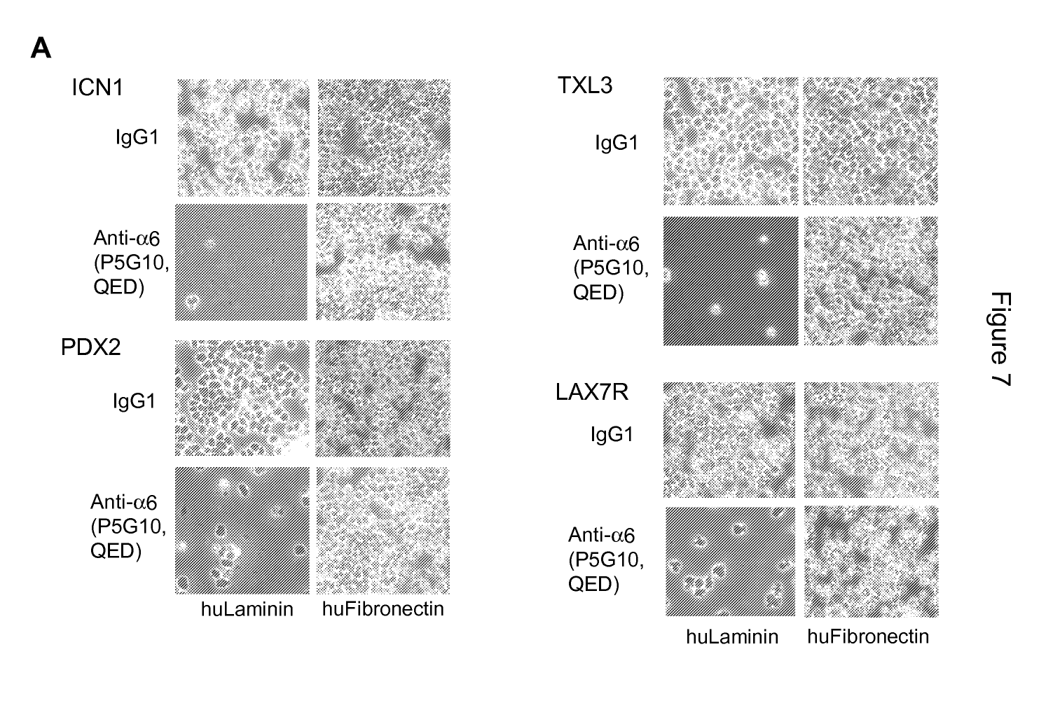

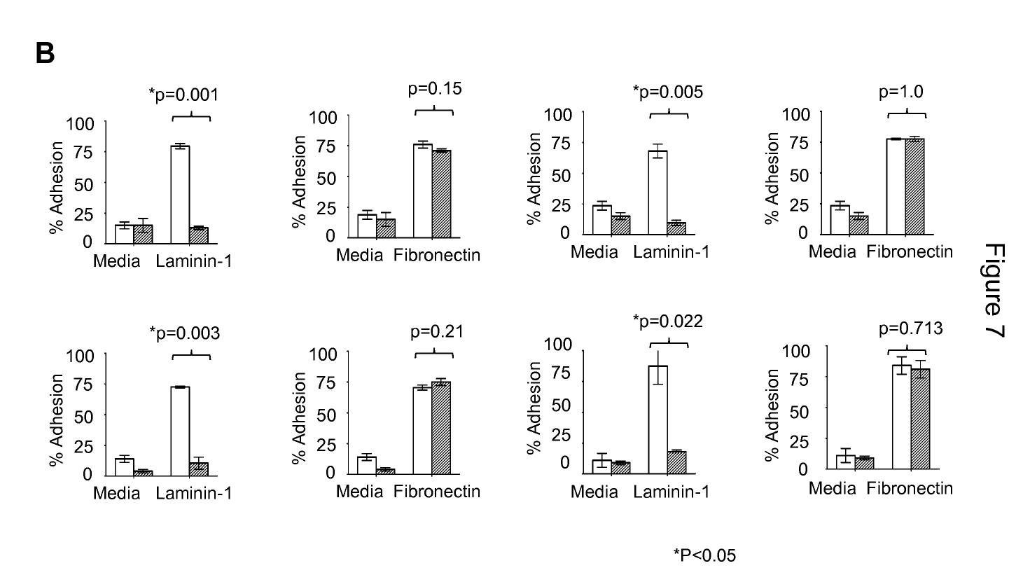

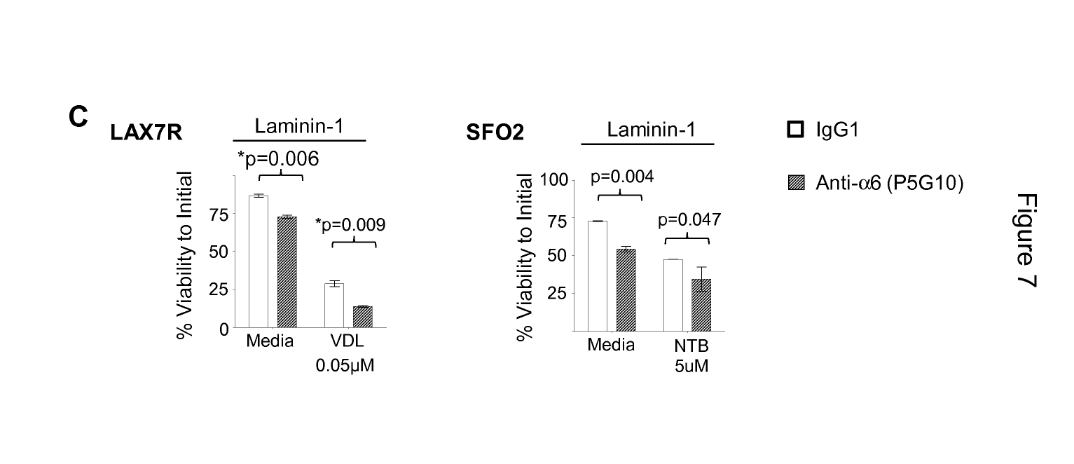

To determine the effect of VLA6 deletion on leukemia progression in vivo, ITGA6 competent ITGA6fl/fl BCR/ABL1+ pre-B (B220+CD19+) CreERT2+ or control transduced ALL cells were lentivirally luciferase labeled and transferred into NOD/SCID mice (FIG. 6A). 3 days thereafter, ITGA6 deletion was induced by Tamoxifen administration to all animals in 2 cycles for 5 days (FIG. 6B). In vivo deletion of ITGA6 delayed leukemia progression compared to ITGA6 competent controls from a median survival time (MST) of 27 days post-leukemia injection to a MST of 54.5 days post-leukemia injection (p=0.0021 Log-rank test) (FIG. 6B). Bioluminescent imaging also showed a significant delay in progression of ITGA6-deleted leukemia compared to ITGA6-competent cells when ITGA6 (FIG. 6A). In vivo deletion of ITGA6 in combination with Nilotinib significantly prolonged survival after leukemia cell transfer, since recipients of ITGA6 competent cells died of leukemia with an MST of 39.5 days despite Nilotinib treatment, whereas Nilotinib-treated recipients of ITGA6 ablated leukemia survived (p=0.0014) (FIG. 6B). Deletion status of ITGA6 was determined when the animals were sacrificed and showed almost completely deleted ITGA6 (FIGS. 6C, D). Animals were sacrificed on Day 127 post-leukemia injection for analysis of minimal residual disease. For this purpose, splenic cells and bone marrow cells of leukemia recipient animals were isolated and analyzed for BCR-ABL1 mRNA expression by RT-PCR (FIG. 6E) and BCR-ABL1 expression in genomic DNA by PCR (FIG. 6F). Thus, combination of ITGA6 deletion with tyrosine kinase inhibition eradicates murine leukemia.

### Example 8

Integrin ITGA6 Blockade De-Adheres Pre-B ALL cells and Overcomes Drug Resistance

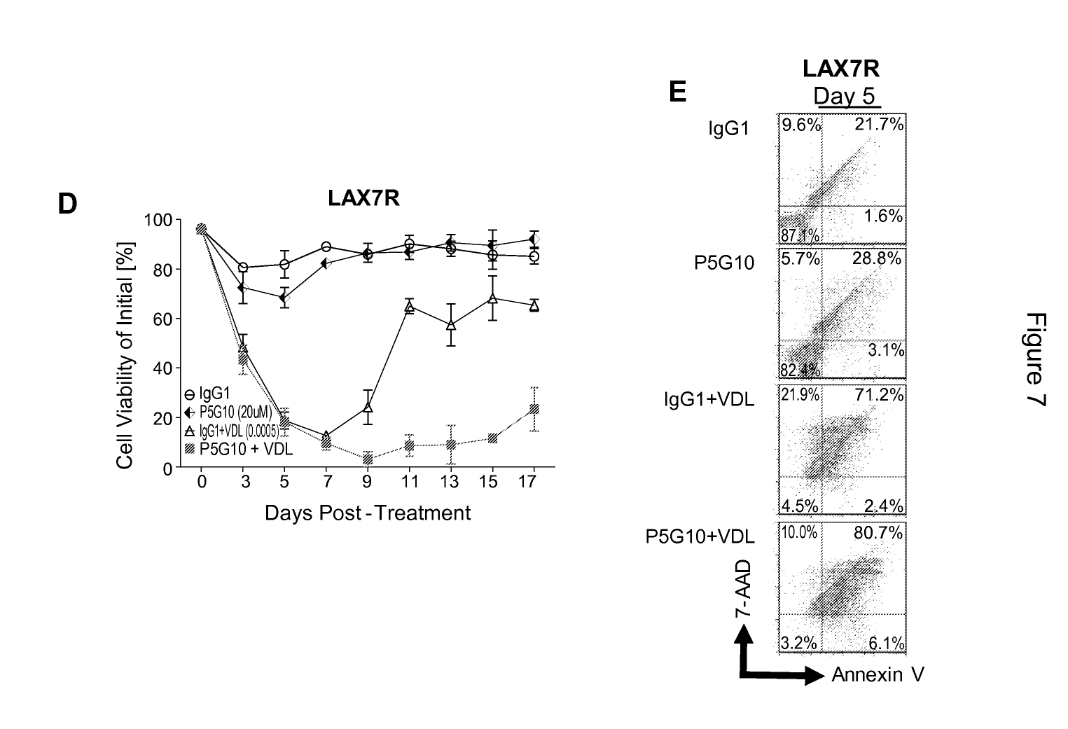

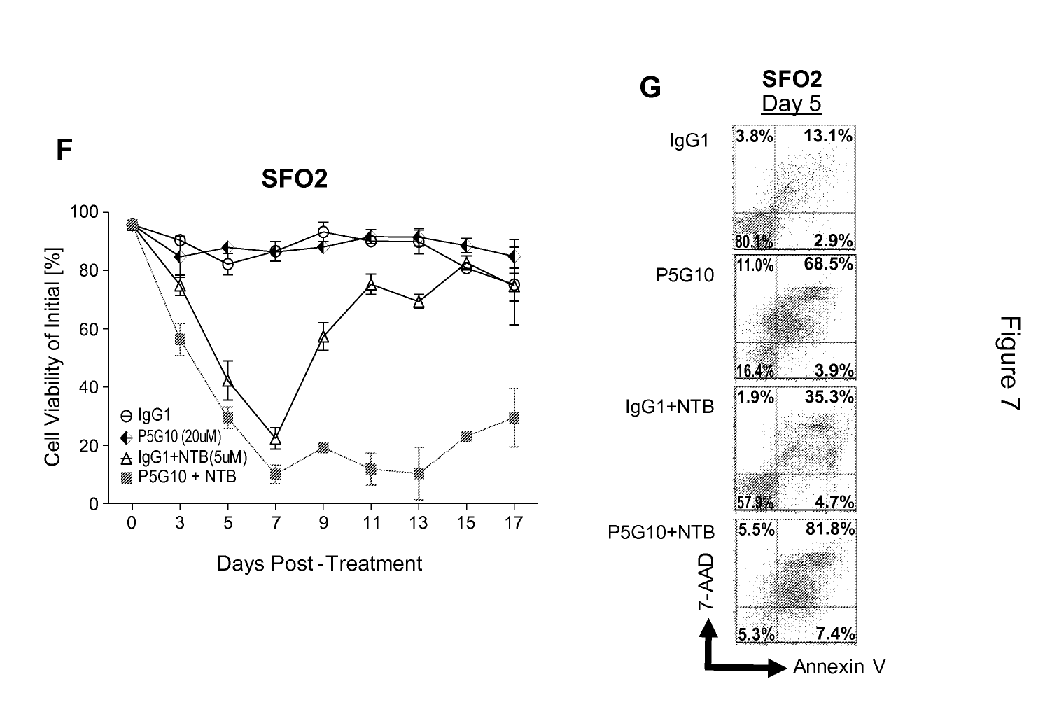

Based on preliminary data supporting that ITGA6 is a central adhesion molecule in bone marrow adhesion-mediated chemoresistance, we evaluated integrin ITGA6 blockade in four patient-derived (primary) ALL cells (ICN1, PDX2, TXL3, LAX7R) using an anti-functional ITGA6 antibody (P5G10) demonstrated to block ligand binding (Reference 30 Wayner & Hoffstrom) with and without the counter ligand laminin-1. ITGA6 blockade de-adhered all four ALL cases from laminin compared to control-treated ALL cells (FIGS. 7A, B) and the percentage of adherence was significantly different. P5G10 did not de-adhere leukemia cells from fibronectin plates indicating the specific blockade of integrin alpha6-laminin-1 interaction. To determine the effect of ITGA6 modulation in chemoresistant ALL, primary ALL cells (PDX2) were treated with a tyrosine kinase inhibitor (TKI), Nilotinib, as PDX3 is Philadelphia chromosome positive (BCR-ABL1+). Primary ALL cells (LAX7R, BCR-ABL negative; SFO2, BCR-ABL+) showed decreased viability after monotreatment with P5G10 (FIG. 7C) and were sensitized when ITGA6 blockade was combined with chemotherapy or TKI, compared to TKI monotreatment. Critically, in a long term co-culture assay of primary ALL cells with murine OP9 cells, alpha6 blockade in combination with VDL (FIGS. 7D, E) or NTB (FIGS. 7F, G) led to marked decrease in viability of ALL cells compared to VDL or NTB treatment.

### Example 9

### ITGA6 Blockade Does Not Mobilize Leukemia Cells to the Peripheral Blood, Sensitizes Leukemia Cells to Chemotherapy and Eradicates Leukemia In Vivo

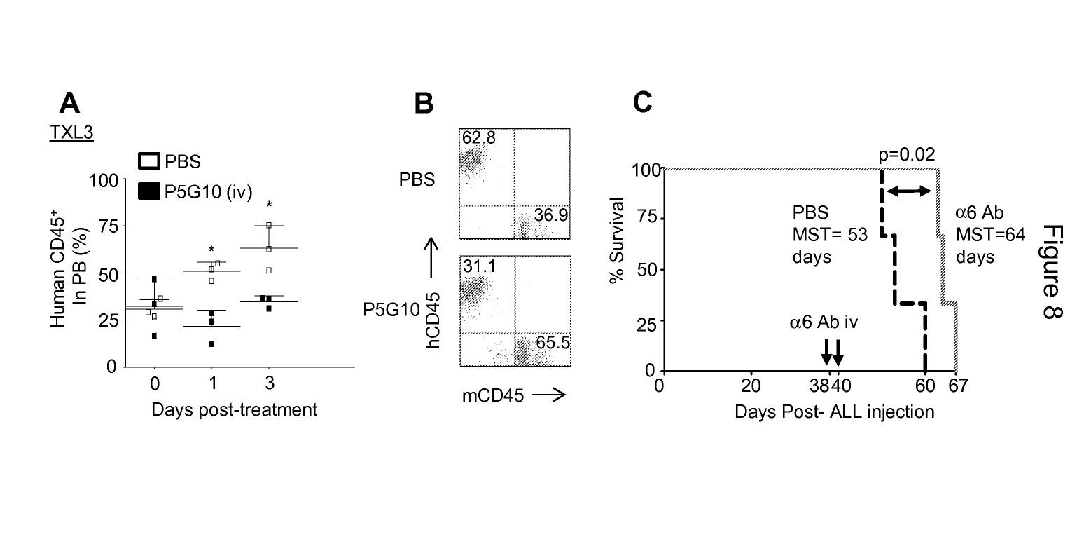

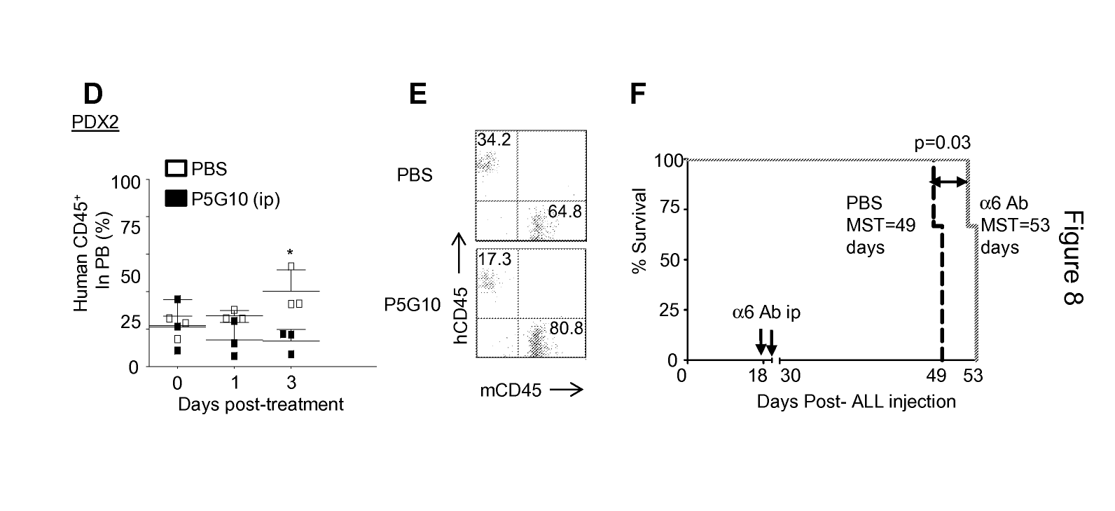

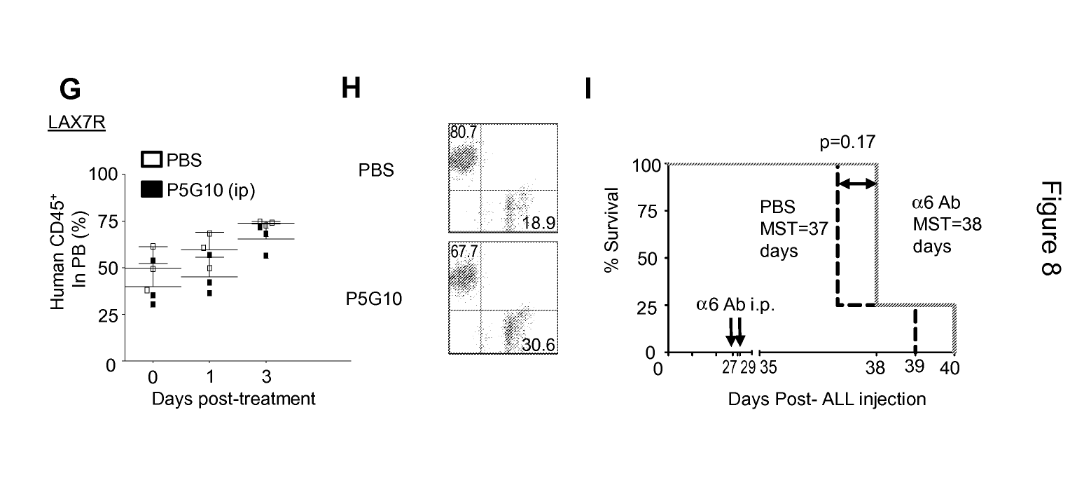

To determine, if ITGA6 blockade induces mobilization of leukemia cells to the peripheral blood, patient-derived ALL cells, TXL3 and PDX2 (both BCR-ABL1+), and LAX7R (G-I) (BCR-ABL negative) were injected into NSG mice. Recipient mice were then treated with 30 mg/kg P5G10 or PBS control by i.v. or i.p. injection after determination of engraftment of leukemia by flow cytometry of human CD45. The % of human CD45+ and CD19+ in peripheral blood (PB) was analyzed by flow cytometry before (pre), 1 and 3 days after (post) mobilization treatment with P5G10 (Tx). In all three cases, we did not observe an increase of leukemia cells in the peripheral blood compared to before P5G10 treatment (Day 0) or compared to the control recipient mice (FIGS. 8A, D, G). Analysis of the Kaplan-Meier survival curve showed in increased prolongation of P5G10 monotreated NSG mice compared to the control treated mice (FIGS. 8C, F, I) (p<0.05, Log-rank Test, n=3/group).

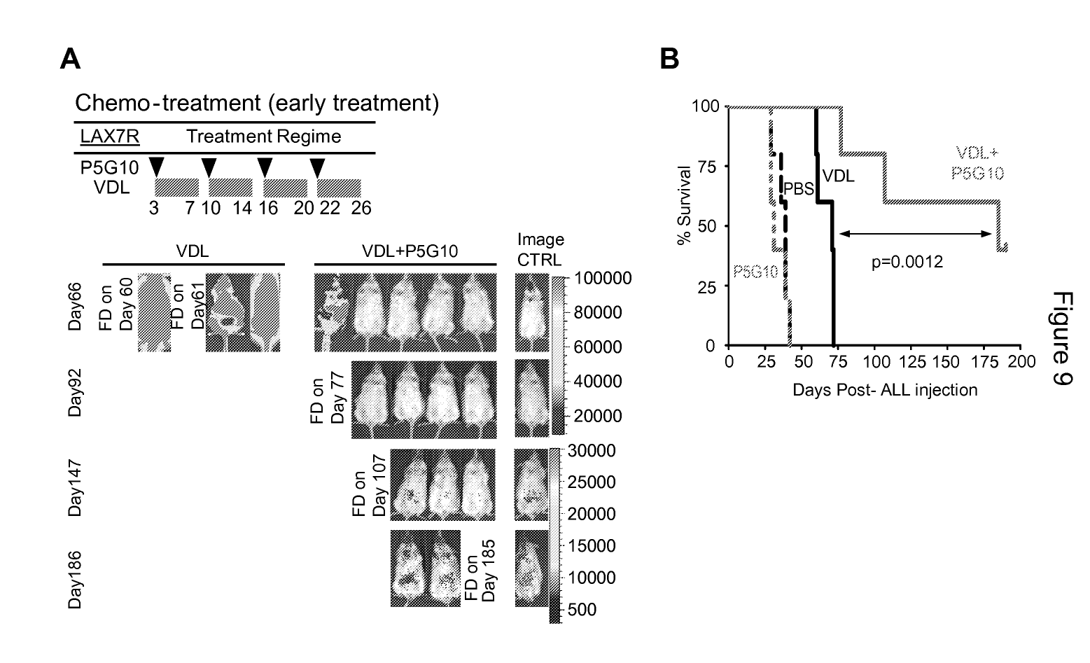

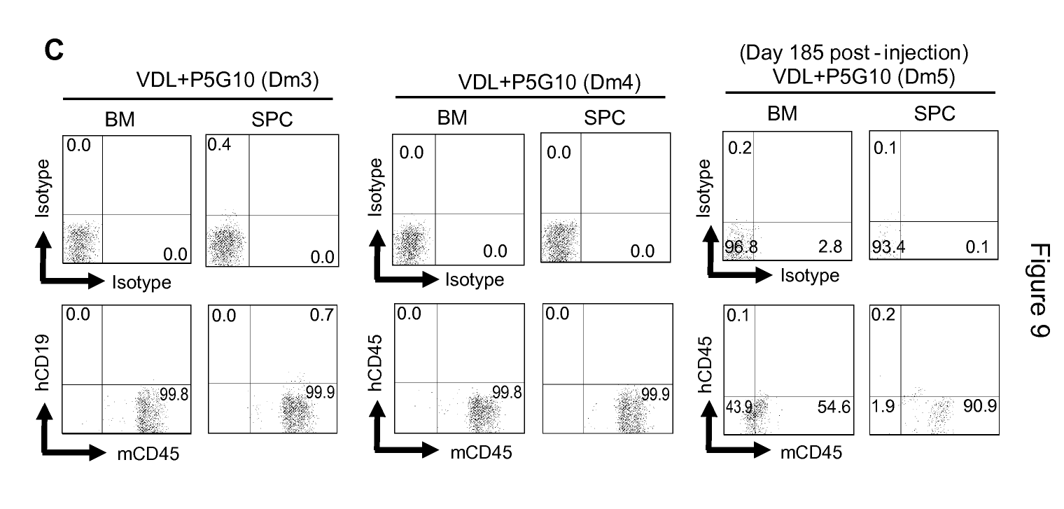

Next, we determined, if P5G10 can restore chemosensitivity of leukemia cells in vivo. For this purpose, we injected luciferase-labeled LAX7R cells into NOD/SCID mice. Three days after leukemia cell injection leukemia cell-bearing mice received four weekly injections of 30 mg/kg blocking alpha6 mAb (P5G10, a non-humanized IgG1) or saline±VDL (FIG. 9A). Mice treated with P5G10 survived similarly as control mice (PBS: MST=39 days vs. P5G10: MST=31 days; p=0.05). Chemotherapy-treated mice relapsed shortly after the end of the four-week treatment and succumbed to leukemia) (MST=71 days) (FIG. 9B). In marked contrast, mice treated with chemotherapy plus P5G10 survived disease-free (MST=185 days; p=0.004). The combination treated animals were found dead or sacrificed Day 185 or Day 186 post-leukemia injection and analyzed for expression of human CD19 or CD45 by flow cytometry, which was not observed (FIG. 9C).

### REFERENCES

1. Pui, C. H., et al. Treating childhood acute lymphoblastic leukemia without cranial irradiation, N. Engl. J. Med., 360: 2730-2741, 2009.
2. Faderl, S., et al. Adult acute lymphoblastic leukemia: concepts and strategies, Cancer, 116: 1165-1176, 2010.
3. Larson, S. and Stock, W. Progress in the treatment of adults with acute lymphoblastic leukemia, Curr. Opin. Hematol., 15: 400-407, 2008.
4. Gaynon, P. S., et al. Children's Cancer Group trials in childhood acute lymphoblastic leukemia: 1983-1995, Leukemia, 14: 2223-2233, 2000.
5. Ross, M. E., et al. Classification of pediatric acute lymphoblastic leukemia by gene expression profiling, Blood, 102: 2951-2959, 2003.
6. van Zelm, M. C., et al. Ig gene rearrangement steps are initiated in early human precursor B cell subsets and correlate with specific transcription factor expression, J. Immunol., 175: 5912-5922, 2005.
7. Georges-Labouesse, E., et al. Absence of integrin alpha 6 leads to epidermolysis bullosa and neonatal death in mice, Nat. Genet., 13: 370-373, 1996.
8. Pear, W. S., et al. Efficient and rapid induction of a chronic myelogenous leukemia-like myeloproliferative disease in mice receiving P210 bcr/abl-transduced bone marrow, Blood, 92: 3780-3792, 1998.
9. Park, E., et al. Targeting survivin overcomes drug resistance in acute lymphoblastic leukemia, Blood, 118: 2191-2199, 2011.
10. Kang, H., et al. Gene expression classifiers for relapse-free survival and minimal residual disease improve risk classification and outcome prediction in pediatric B-precursor acute lymphoblastic leukemia, Blood, 115: 1394-1405, 2010.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1. A method for treating a hematological malignancy in a subject in need thereof comprising:
(a) providing a composition comprising an inhibitor of integrin alpha 6;
(b) administering an effective amount of the inhibitor so as to treat cancer in the subject.

2. A method for treating a solid tumor in a subject in need thereof comprising:
(a) providing a composition comprising an inhibitor of integrin alpha 6;
(b) administering an effective amount of the inhibitor so as to treat cancer in the subject.

3. (canceled)

4. The method of claim 1, wherein the hematological malignancies are any one or more of leukemia, lymphoma, Chronic Myeloproliferative Disorders, Langerhans Cell Histiocytosis, Multiple Myeloma/Plasma Cell Neoplasm, Myelodysplastic Syndromes, Myelodysplastic/Myeloproliferative Neoplasms or a combination thereof.

5. The method of claim 4, wherein leukemia is any one or more of Acute Lymphoblastic Leukemia (ALL), Acute Myeloid Leukemia (AML), Chronic Lymphocytic Leukemia (CLL), Chronic Myelogenous Leukemia (CML), Hairy Cell Leukemia (HCL) or a combination thereof.

6. The method of claim 4, wherein lymphoma is any one or more of AIDS-Related Lymphoma, Cutaneous T-Cell Lymphoma, Hodgkin Lymphoma, Mycosis Fungoides, Non-Hodgkin Lymphoma, Primary Central Nervous System Lymphoma, Sézary Syndrome, T-Cell Lymphoma, Cutaneous, Waldenström Macroglobulinemia or a combination thereof.

7. The method of claim 2, wherein a solid tumor is any one or more of Adrenocortical Tumors (Adenoma and Carcinoma), Carcinoma, Colorectal Carcinoma, Desmoid Tumors, Desmoplastic Small Round Cell Tumor, Endocrine Tumors, Ewing Sarcoma, Germ Cell Tumors (Solid Tumor), Hepatoblastoma, Hepatocellular Carcinoma, Melanoma, Neuroblastoma, Osteosarcoma, Retinoblastoma, Rhabdomyosarcoma, Soft Tissue Sarcomas Other Than Rhabdomyosarcoma and/or Wilms Tumor

8. The method of claims 1 or 2, wherein the inhibitor is any one or more of peptides, proteins, small molecules, nucleic acids, aptamers, oligonucleotides, antibodies or a combination thereof.

9. The method of claim 8, wherein the antibody is selected from the group consisting of a monoclonal antibody or a fragment thereof, a polyclonal antibody or a fragment thereof, chimeric antibodies, humanized antibodies and single chain antibody.

10. The method of claim 8, wherein antibody is a monoclonal antibody or a fragment thereof.

11. The method of claim 10, wherein the monoclonal antibody is P5G10 or a fragment thereof, P1H8 or a fragment thereof or combinations thereof.

12. The method of claims 1 or 2, wherein the methods further comprise administering any one or more of a chemotherapeutic agent, tyrosine kinase inhibitor, integrin alpha 4 inhibitor or a combination thereof.

13. The method of claim 12, wherein the chemotherapeutic agent, the tyrosine kinase inhibitor, the integrin alpha 4 inhibitor or the combination thereof are administered sequentially or simultaneously with the composition comprising the integrin alpha 6 inhibitor.

14. The method of claim 10, wherein the antibodies are conjugated to a therapeutic agent.

15. A pharmaceutical composition comprising an inhibitor of integrin alpha 6 and a pharmaceutically acceptable carrier.

16. The pharmaceutical composition of claim 14, wherein the inhibitor is any one or more of peptides, proteins, small molecules, nucleic acids, oligonucleotides, antibodies or a combination thereof.

17. The pharmaceutical composition of claim 15, wherein the antibody is selected from the group consisting of a monoclonal antibody or a fragment thereof, a polyclonal antibody or a fragment thereof, chimeric antibodies, humanized antibodies and single chain antibody.

18. The pharmaceutical composition of claim 14, wherein the inhibitor is a monoclonal antibody.

19. The pharmaceutical composition of claim 17, wherein the monoclonal antibody is P5G10, P1H8 or a combination thereof.

20-24. (canceled)
